(19) United States
(12) Patent Application Publication
(10) Pub. No.: US 2006/0035383 A1
(43) Pub. Date: Feb. 16, 2006

(54) Dry platelet preparations for use in diagnostics

(76) Inventors: David Ho, Fairfax, VA (US); Keith A. Moskowitz, Germantown, MD (US); Cindy Orser, McLean, VA (US); Alan Rudolph, Potomac, MD (US)

Correspondence Address: MOAZZAM & LATIMER LLP, 1474 North Point Village Center #320, Reston, VA, 20194-1190, US

(21) Appl. No.: 11/152,774

(22) Filed: Jun. 15, 2005

Related U.S. Application Data
(60) Provisional application No. 60/600,838, filed on Aug. 12, 2004.
(60) Provisional application No. 60/619,930, filed on Oct. 20, 2004.
(60) Provisional application No. 60/671,063, filed on Apr. 14, 2005.

(51) Int. Cl. G01N 31/00; G01N 33/86

(52) U.S. Cl. 436/69; 436/16

(57) ABSTRACT

The present invention provides compositions comprising freeze-dried platelets, microparticles, or both for use as a diagnostic for blood coagulation function. The invention also provides methods of diagnosing or monitoring blood coagulation function, including diagnosing or monitoring blood coagulation diseases and disorders. Kits for performing the methods of the invention are also provided.

### CROSS-REFERENCE TO RELATED APPLICATIONS

This application relies on and claims the benefit of the filing dates of U.S. Provisional patent application No. 60/600,838, filed 12 Aug. 2004, U.S. Provisional patent application No. 60/619,930, filed 20 Oct. 2004, and U.S. Provisional patent application No. 60/671,063, filed 14 Apr. 2005, the entire disclosures of all of which are hereby incorporated herein by reference in their entireties.

### BACKGROUND OF THE INVENTION

1. Field of the Invention

The present invention relates to the field of dry platelet preparations. More specifically, the present invention relates to dry platelet preparations and their use in diagnosis and monitoring of diseases and disorders relating to platelet function.

2. Description of Related Art

Platelets are formed in the bone marrow as fragments of megakaryocytes. They are irregularly-shaped, colorless bodies that are present in blood at a concentration of 150,000-450,000 per microliter (ul). Platelets play a crucial role in normal hemostasis, and they are the first line of defense against blood escaping from injured blood vessels. When bleeding from a wound suddenly occurs, the platelets gather at the wound and attempt to block the blood flow by forming a clot. The sticky platelets adhere to the damaged area and gradually form a platelet plug. At the same time, the platelets release a series of chemical signals that prompt other factors in the blood to reinforce the platelet plug. Between the platelet and its reinforcements, a sturdy clot is created that acts as a patch while the damaged area heals.

Blood clotting is a complicated process: if the clot formation is unchecked, the vessel will become occluded; if the clot is not sturdy, excessive blood loss will occur. Therefore, a delicate balance must be maintained for normal hemostasis. In situation where normal hemostasis is unbalanced, clot formation may be compromised. Such an abnormality could be acquired due to injestion of aspirin or caused by immune dysfunction. The abnormality could also be congenital, such as through genetic diseases and clotting factor defects. For example, defects in the process of hemostasis that lead to bleeding disorders have been identified, and most of such defects are in the enzymes involved in the cascade of activities required for clotting, in platelet activation and function, or in contact activation. Included among these disorders are vWD and hemophilia. Other diseases or disorders of the blood clotting system are a result (i.e., side effect) of treatments for other diseases or disorders. Treatments for such diseases and disorders typically involve reducing the dose of the drug causing the side effect, or discontinuing treatment with the drug.

Typically, detection of a blood clotting disease or disorder involves analyzing the patient's blood for platelet counts, various markers involved in blood clotting, and clot-forming ability. The coagulation assays measured the activated clotting time (ACT), the prothrombin time (PT), the plasma thrombin time (PTT), and the activated partial thromboplatin time (APTT) are used to evaluate the intrinsic and extrinsic coagulation pathways. These assays are generally performed in the laboratory and analysis often requires multiple samples of blood to be drawn from the patients. Moreover, these assays are potentially unreliable as they are end-point tests in which results are based on the time of clot formation in vitro. Another limitation relates to the fact that exogenous reagents, such as kaolin, thrombin, calcium, etc. must be added thus, the results are based on an artificial system, and do not necessarily reflect the patent's thrombotic potential.

Platelet functionality is another critical component of blood clots. Dysfunctional platelets may lead to abnormal hemorrhage, such as bleeding or thrombosis. Thus platelet function assays are an integral part of the diagnosis and monitoring of blood related diseases. For example, acquired platelet defects, such as injestion of aspirin, cardiac disease, renal disease, or congenital platelet defects such as Bernard-Soulier syndrome, Glanzmann's thrombasthenia and storage pool disease, to name a few, can influence the normal hemostatic function of the platelets. To assess the platelet function, at the very minimal, a complete blood count with a peripheral blood smear will provide some basic information. Other tests, such as bleeding time, platelet function tests using an aggregometer to assess the aggregation of platelets to a panel of platelet agonists performed on whole blood or platelet rich plasma will classify the defect. However, such analyses, although accurate, are not highly sensitive, and can fail to detect slight perturbances in normal clotting function at early stages of a disorder. Likewise, determination of the precise point of failure of the blood clotting cascade may require numerous assays using freshly drawn blood.

Even though numerous advances in detecting and treating bleeding disorders have taken place over the last several years, there is still a need for improved compositions and methods for detecting such disorders, particularly more sensitive and accurate methods to detect the development of the disorders.

### SUMMARY OF THE INVENTION

The current invention addresses needs in the art by providing compositions and methods that can be used as diagnostics for detection of blood clotting disorders. The compositions can be produced following the methods provided herein, and can contain platelets, microparticles, such as platelet-derived microparticles, or both. Accordingly, the present invention provides methods of making diagnostic compositions and using them in methods of diagnosing bleeding disorders. Kits comprising the diagnostic compositions are also provided.

In a first aspect, the present invention provides compositions comprising platelets. The platelets can be freeze-dried, reconstituted from freeze-dried platelets, or fresh. The compositions can, but do not necessarily, comprise microparticles in addition to the platelets. The compositions can be used to diagnose a disorder of the blood clotting system. They likewise can be used to monitor the blood clotting ability of a patient's blood clotting system over a period of time, such as, for example, during a treatment regimen for a disease or disorder of the blood clotting system or another system or tissue within the patient's body.

In a second aspect, the invention provides methods of making the compositions of the invention. In general, the methods comprise obtaining platelets and freeze-drying them. The methods can further comprise adding the freeze-dried platelets to other platelets or to plasma, to form a mixture. Freeze-dried platelets according to the present invention, alone or in conjunction with other platelets and plasma, are useful for diagnosing various diseases and disorders of the blood clotting system. The platelets of the compositions may be indated (freshly isolated) or outdated (older than permitted by FDA regulations for therapeutic uses of blood).

In a third aspect, the present invention provides methods of diagnosing a disease or disorder of the blood clotting system. The methods generally comprise obtaining freeze-dried platelets, combining the freeze-dried platelets with platelets and/or plasma removed from a patient having, or suspected of having, a disease or disorder of the blood clotting system to form a mixture, and determining whether the person has a defect in the blood clotting system by assaying one or more biological or biochemical functions of the mixture, where the defect decreases or abolishes the patient's blood clotting system's ability to function normally or to cause clot formation in a pre-defined period of time. Typically, determining whether the patient's blood clotting system is defective comprises assaying clotting time of the mixture.

In a fourth aspect, the invention provides methods of monitoring the progression of a disease or disorder of the blood clotting system. The methods generally comprise obtaining freeze-dried platelets, combining the freeze-dried platelets with platelets and/or plasma removed from the patient suffering from the disease or disorder to make a mixture, and determining the blood clotting ability of the mixture. Typically, determining the blood clotting ability of the mixture indicates the blood clotting ability of the patient's blood, and comprises assaying clotting time of the mixture. Furthermore, typically, multiple assays are performed over time to give an indication of progression over time.

In a fifth aspect, the invention provides methods of monitoring the effects of a treatment regimen for a patient on the blood clotting system of that patient. In general, the methods comprise obtaining freeze-dried platelets, combining the freeze-dried platelets with platelets and/or plasma removed from the patient undergoing the treatment regimen to make a mixture, and determining the blood clotting ability of the mixture. Typically, determining the blood clotting ability of the mixture indicates the blood clotting ability of the patient's blood, and comprises assaying clotting time of the mixture. Furthermore, typically, multiple assays are performed over time to give an indication of the effects of the treatment regimen over time.

In a sixth aspect, the invention provides kits for performing the methods of the invention. Typically, the kits of the invention comprise freeze-dried platelets. The kits can also comprise some or all of the other reagents and supplies necessary to perform at least one embodiment of one method of the invention. Thus, the kits can be diagnostic kits, blood clotting monitoring kits for coagulation proteins or platelets, or drug treatment monitoring kits. Often, the kits will comprise some or all of the supplies and reagents to perform one or more control reactions to ensure the kits are performing properly and to provide baseline results against which test samples can be compared.

### BRIEF DESCRIPTION OF THE DRAWINGS

The accompanying drawings, which are incorporated in and constitute a part of this specification, illustrate several embodiments of the invention, and together with the written description, serve to explain principles of the invention.

FIG. 1 depicts the size distribution of rehydrated freeze-dried platelets, a composition of the invention, and freshly isolated platelets.

FIG. 2 depicts a standard curve of freeze-dried platelets vs. clotting time using normal pooled plasma.

FIG. 3 depicts a standard curve of freeze-dried platelets vs. clotting time using platelet-poor plasma.

FIG. 4 depicts detection of clotting defects in hemophilia plasma.

FIG. 5 depicts a general schema for coagulation and inhibitors of coagulation.

FIG. 6 depicts results of assays distinguishing coagulation protein defects in whole blood.

FIG. 7 depicts specific reaction of freeze-dried platelets of the invention with anti-coagulants.

FIG. 8 shows that freeze-dried platelets are activated with ionophores, which expose additional binding sites for FITC-Annexin V binding to freeze-dried platelets.

FIG. 9 shows that freeze-dried platelets binding to 50 nM FITC-Annexin V can be competed with 100 fold excess of unlabled Annexin V.

FIG. 10 shows that 25 nM of labeled FVIIa failed to bind to both unactivated and inonophore activated fresh platelets.

FIG. 11 depicts direct binding of 25 nM of FVIIa to freeze-dried platelets and shows that the binding can be competed off using 2500 nM unlabeled FVIIa.

FIG. 12 depicts direct binding of 100 nM of FXa to freeze-dried platelets and shows that the binding can be competed off using 10000 nM unlabeled Fxa.

FIG. 13 depicts the effects on collagen-mediated aggregation of freeze-dried platelets, fresh platelets, and combinations of the two.

FIG. 14 depicts the effects of collagen-mediated aggregation as judged by single cell count of freeze-dried platelets, fresh platelets, and combinations of the two.

FIG. 15 depicts the effect on freeze-dried platelets when exposed to arachidonic acid, collagen, epinephrine, thrombin receptor activating peptide (TRAP), and ristocetin mediated aggregation of freeze-dried platelets. The figure also depicts the percent aggregation of freeze-dried platelets as judged by single cell count.

### DETAILED DESCRIPTION OF CERTAIN EMBODIMENTS OF THE INVENTION

The present invention recognizes, for the first time, the usefulness of freeze-dried platelets in detecting and monitoring diseases and disorders affecting the blood clotting system. It also recognizes, for the first time, the usefulness of freeze-dried platelets in monitoring the effects of drugs and drug treatment regimens on the blood clotting system of individuals to whom the drugs are administered. In essence, it recognizes that freeze-dried platelets are suitable for all diagnostic capabilities provided by fresh platelets, including monitoring any and all functions of platelets. It thus recognizes the usefulness of freeze-dried platelets in monitoring the blood clotting ability of an individual's blood. The discovery that freeze-dried platelets can be used as a substitute for fresh platelets in various assays enables methods of monitoring the blood clotting ability of blood samples, and provides information that can be important or critical to the health and life of individuals.

In a first aspect, the present invention provides compositions comprising platelets. The compositions can consist of platelets, or can comprise any number of substances in addition to platelets. Thus, a composition of the invention can be a solid or a liquid. When in the form of a liquid, the composition can comprise water or another aqueous solvent, such as an aqueous buffer, blood or a blood component or fraction, saline, buffered saline (e.g., phosphate buffered saline), or the like. The liquid can also comprise one or more organic solvents, such as one or more alcohols.

In various embodiments of the invention, the composition comprises platelets that are freeze-dried, or derived from freeze-dried platelets. Thus, in embodiments, the composition consists of freeze-dried platelets. In other embodiments, the composition comprises platelets that are derived from freeze-dried platelets, such as, for example, platelets that were freeze-dried then reconstituted with water, saline, or plasma (also referred to herein as reconstituted or rehydrated platelets).

The compositions comprising platelets can comprise one or more other substances in addition to the platelets. For example, they may comprise one or more substances that were present with the platelets before, during, or after the platelets were freeze-dried. Thus, the compositions comprising platelets can also comprise one or more salts, such as phosphate salts, sodium salts, potassium salts, calcium salts, magnesium salts, and any other salt that can be found in blood or blood products, or that is known to be useful in freeze-drying platelets or eukaryotic cells, or any combination of two or more of these. Other exemplary substances that may be present in the compositions include, but are not limited to, sugars, such as monosaccharides and disaccharides (e.g., maltose, dextrose, mannose, trehalose, sucrose, polymers of sucrose, glucose), glycerol, triglycerides, polysaccharides, lipids, and the like. Yet other exemplary substances include biological molecules derived from human or animal sources, such as albumin, casein, laminin, fibrinogen, and the like. Of course, because the freeze-drying procedure can result in lysis of a certain number of platelets, compositions of the invention may comprise, external to intact platelets, some or all of the components present in the interior of a platelet.

One particular group of substances that may be present in a composition of the invention is chemical and biological compounds that function as drugs. Another group is substances that function as food. Yet another group is substances that function as herbal supplements. In embodiments, the substances are anti-coagulants. As will be discussed below in more detail, the compositions and methods of the present invention are particularly well suited for detection and monitoring of drugs, food, and herbal supplements in blood samples, and detection and monitoring of the effects of these substances on the blood clotting system of the patient to whom the drugs, etc. are administered. Among the drugs are Warafin (Coumadin®), Heparin, Clopidogrel (Plavix®), Dipyridamole (Persantine®), Enoxaparin (Lovenox®), Ardeparin (Normiflo®), Dalteparin (Fragmin®), Ticlopidine (Ticlid®), Danaparoid (Orgaran®), Tinzaparin (Innohep®), Aspirin, Thrombin Inhibitors, and the like. Also among the substances are certain food and herbal supplements that contain coumarins with potential anticoagulant effects, such as Alfalfa, Angelica (Don Quai), Arnica, Bogbean, Capsicum, Celery, Dandelion, Horse chestnut, Horseradish, Meadowsweet, Nettle, Parsley, Passion, Flower, Red Clover, Sweet Clover, Wild Carrot, Wild Lettuce. In addition, the substances can be those that have anti-platelet properties, such as Agrimony, Aloe gel, Black cohosh, Bogbean, Clove, Dandelion, Garlic, Ginger, Ginkgo biloba, Ginseng (Panax), Licorice, Meadowsweet, Onion, Policosanol, Poplar, Senega, Tamarind, Willow Wintergreen, and the like.

It has been found that freeze-drying of platelets using the methods of the present invention creates varying levels of microparticles. Thus, in embodiments, the compositions of the present invention comprise platelets and microparticles. In such embodiments, the platelets typically comprise about 10% to about 70% of the total number of particles in the composition. For example, platelets can comprise about 10% to about 60% of the particles, about 20% to about 50% of the particles, or about 20% to about 30% of the particles. In exemplary embodiments, the composition comprises platelets and microparticles as essentially the only particles that are part of the blood clotting system, and comprises platelets in an amount of about 10%, about 20%, about 30%, about 40%, about 50%, or about 60% of the total particle count. Of course, a composition of the invention may comprise any specific percentage number, or fraction thereof, of platelets within the ranges discussed above.

It is to be noted at this point that each value stated in this disclosure is not, unless otherwise stated, meant to be precisely limited to that particular value. Rather, it is meant to indicate the stated value and any statistically insignificant values surrounding it. As a general rule, unless otherwise noted or evident from the context of the disclosure, each value includes an inherent range of 5% above and below the stated value. At times, this concept is captured by use of the term “about”. However, the absence of the term “about” in reference to a number does not indicate that the value is meant to mean “precisely” or “exactly”. Rather, it is only when the terms “precisely” or “exactly” (or another term clearly indicating precision) are used is one to understand that a value is so limited. In such cases, the stated value will be defined by the normal rules of rounding based on significant digits recited. Thus, for example, recitation of the value “10” means any whole or fractional value between 9.5 and 10.5, whereas recitation of the value “exactly 100” means 99.5 to 100.4.

The platelets that are present in compositions of the invention have essentially all of the gross morphological characteristics of normal, freshly obtained platelets in blood. For example, in certain compositions where freeze-dried platelets are present, about 70% of the particles in the composition are retained when the composition is filtered through a mesh size that retains particles of the size of a typical platelet. Likewise, generally the platelet particles show the same array of cell surface proteins as fresh, untreated platelets. For example, size, granularity, and surface receptors, such as GPIb and GPIIb/IIIa, are retained or partially retained on the surface of the freeze-dried platelets at the levels comparable to fresh platelets. The composition of the invention also contains characteristic that are not commonly found in fresh platelets, such as expression of charged lipids and granule proteins, such as P-selectin and Factor V. Due to this, the composition confers addition functions that fresh platelets can not perform, such as binding to Vitamin K-dependent proteins and the like.

The compositions of the invention can comprise platelets from any source. That is, the compositions can comprise platelets from any mammalian species, including, but not limited to, humans, primates, canines, felines, bovines, ovines, porcines, equines, and rodents. They therefore can be platelets from a human, chimp, dog, cat, cow, sheep, pig, horse, mouse, or rat. In addition, the platelets can be autologous or heterologous, with respect to the blood with which they are mixed in the methods of the invention. More specifically, the methods of the invention generally comprise mixing platelets, such as freeze-dried platelets, with freshly obtained blood from a patient. The platelets are preferably, but not necessarily, obtained from the same patient as the blood (i.e., autologous platelets). However, in embodiments, the platelets are obtained from one or more individuals other than the patient (i.e., heterologous platelets). In certain embodiments, the freeze-dried platelets originate from a pool of platelets obtained from two or more donors. In certain embodiments that relate to compositions comprising both freeze-dried platelets and fresh platelets, the fresh platelets originate from a pool of platelets obtained from two or more donors.

Platelets for use in the invention can be obtained from indated or outdated blood. Indated blood is blood that has freshly been obtained from a donor, and includes blood that is less than six days old. In contrast, outdated blood is blood that was obtained from a donor six or more days earlier, and thus is no longer deemed by governmental regulatory agencies as suitable for use as a therapeutic agent to treat excessive bleeding (e.g., for blood transfusions). In certain embodiments, outdated blood from one or multiple donor sources (used singly or as a mixture of blood from different sources) is used as a source of freeze-dried platelets to be used as a “normal” or “standard” control.

The freeze-dried platelets are present in the compositions in an amount of from 1×10⁵ to 1×10¹¹. In embodiments where fresh platelets are present in the compositions, the fresh platelets are present in an amount of from 1×10⁵ to 1×10¹¹. In exemplary embodiments, one or both type of platelets are present in a composition in amounts of about 1×10⁸ to 1×10⁹, such as about 1×10⁹. In compositions comprising both fresh and freeze-dried platelets, the amounts of each may be the same or different. When in a liquid composition, the platelets are present at concentrations ranging from 1×10⁵ per milliliter (ml) to 1×10¹¹ per ml. In exemplary embodiments, the platelets are present in amounts of about 1×10⁸ per ml to 1×10¹⁰ per ml, such as about 1×10⁹ per ml.

As discussed in detail below, the methods of the invention generally comprise mixing freeze-dried platelets with fresh blood or a fraction of fresh blood (e.g., plasma), which might or might not contain platelets, to make a mixture. Such a mixture is considered a composition according to the present invention. Thus, in embodiments, a composition of the invention comprises fresh platelets, which have been obtained from a donor and not subjected to any freeze-drying technique. Likewise, a composition of the invention can comprise a combination of both fresh platelets and freeze-dried platelets. Each of these types of platelets may be present in the composition in any amounts or concentrations, regardless of the amount or concentration of the other. Suitable amounts of each may be selected by the practitioner based, at least in part, on the considerations described herein with regard to practice of the methods of the invention.

The pH of the composition may be any pH that is suitable for stability and function of platelets. Accordingly, it can range from mildly acidic to mildly basic, such as from pH 4.0 to pH 8.5. In various embodiments, the pH of the composition is 4.0, 4.5, 5.0, 5.5, 6.0, 6.5, 7.0, 7.5, 8.0, or 8.5. In other embodiments, the pH is any other pH within the range of 4.0 to 8.5. In embodiments where the platelets are in a solid (dry) state, the compositions may comprise one or more substance that, when hydrated, causes the pH of the resulting liquid composition to be in a suitable range.

Trehalose can be included in the composition, and the trehalose can be present outside the platelets, inside the platelets, or both. Although any amount may be suitable, the amount of trehalose typically ranges from 50 mM to 150 mM. In various embodiments, the trehalose concentration is 50 mM, 75 mM, 100 mM, 125 mM, or 150 mM. In other embodiments, the trehalose concentration is any other concentration within the range of 50 mM to 150 mM. In embodiments where the platelets are in a solid (dry) state, the compositions may comprise one or more substance that, when hydrated, causes the concentration of trehalose of the resulting liquid composition to be in a suitable range.

A composition that is suitable for loading trehalose into platelets can comprise ethanol. In such a composition, the ethanol can range from 0.1% to 5.0% (v/v). In various embodiments, the ethanol concentration is 0.1%, 0.5%, 1%, 2.5%, or 5%. In other embodiments, the ethanol concentration is any other concentration within the range of 0.1% to 5%.

In embodiments where the platelets are in a solid (dry) state, the composition can be heated, such as at room temperature, 50° C., 55° C., 60° C., 65° C., 70° C., 75° C., 80° C., 85° C., or 90° C. In embodiments, the temperature is any temperature within the range of room temperature to about 90° C. The heating process can promote formation of platelets that are suitable for assays of platelet function.

In embodiments where the platelets are in a solid (dry) state, the composition can be heated from less than one minute up to 24 hours or more. Accordingly, the time of heating can be 0, 2, 4, 8, 12, or 24 hours. In other embodiments, the time of heating is any time within the range of less than 1 minute to 24 hours, including any minute or fraction thereof within that range.

As should be evident from the present disclosure, with the exception of certain drugs and anti-platelet compounds, any and all substances that are present in the compositions of the invention are preferably present in amounts that are compatible with at least one function of normal platelets. That is, the compositions of the invention may comprise numerous substances in addition to platelets, but each substance, and the total combination of substances, present is preferably present in an amount that permits the platelets to function normally, at least with respect to one platelet function. In embodiments where one or more substance is present in an amount that inhibits normal platelet function, it is preferred that the substance be removed or adjusted in concentration prior to use of the platelets in a method of the invention in order to permit the method to function well. Of course, these considerations are not relevant to drugs and other anti-platelet substances that are intentionally included in the compositions to determine the effect of such substances on platelet or clotting system function.

In a second aspect, the invention provides a method of making a composition of the invention. In general, the method of this aspect of the invention comprises obtaining platelets and freeze-drying them.

In certain embodiments, the method of making the compositions comprises providing a material that contains platelets and/or microparticles, removing all or essentially all red and white blood cells that might be present in the material, adjusting the pH of the resulting cell-free material to an acidic pH, separating platelets, microparticles, or both from all or essentially all other components present in the material, resuspending the platelets, microparticles, or both in a liquid, and lyophilizing. In embodiments, one or more agents that are typically included in lyophilization procedures, such as sugars, are added to the resuspended platelets and/or microparticles before lyophilizing. Exemplary sugars include, but are not limited to, monosaccharides, disaccharides (e.g., sucrose, lactose, maltose, isomaltose, cellobiose, and trehalose), or polysaccharides. In embodiments, the method comprises sterilizing the lyophilized material using any known technique that is suitable for sterilizing lyophilized materials, including, but not limited to, irradiation.

In a basic procedure for making a composition of the invention, platelets are suspended in a buffer comprising trehalose to give a concentration of about 1×10⁹/ml. The composition is incubated at ambient temperature (about 20° C.-25° C.) for two hours, at which time, 5% bovine serum albumin or any other bulking proteins (such as Casein), is added (final concentration) is added (final concentration), and the platelets are lyophilized using a standard lyophilization protocol. Alternatively, in another basic procedure, 6.0% carbohydrates that can replace proteins as the bulking reagent, such as Ficoll-400 or any other bulking carbohydrate (such as hydrogels), is added (final concentration) and the platelets are lyophilized using a standard lyophilization protocol.

In accordance with the discussion above, the platelets can be obtained from any suitable source. They can be, for example, human platelets, monkey platelets, dog platelets, cat platelets, horse platelets, cow platelets, sheep platelets, goat platelets, pig platelets, rabbit platelets, mouse platelets, or rat platelets. The platelets can be indated or outdated, and can be autologous or heterologous (with regard to the platelets with which they are to be mixed in a method of the invention, discussed below). Accordingly, they can be from random donors units or aphereisis units. The amount of platelets can be any suitable amount, such as those described above.

The step of obtaining can comprise any activity that results in removal of platelets from a donor's body and transfer of the platelets into a receiving vessel. Numerous techniques for achieving this result are known in the art, and any method or combination of methods is encompassed by the present invention. In certain embodiments, obtaining comprises drawing blood from a donor's vein and placing the drawn blood in a tube, such as one made of plastic or glass.

In embodiments, the platelets are obtained from one or more blood donors, and are present in whole blood. It is preferable, however, that the platelets be purified, at least to some extent, from one or more other blood components. This is particularly so for freeze-dried platelets. Methods of purifying or isolating platelets from other blood components are well known to those of skill in the art, and thus need not be detailed here. In exemplary embodiments, platelets are purified from other blood components through a process that comprises centrifugation.

Freeze-drying (also known in the art as lyophilization) can be accomplished by any technique that is suitable for freeze-drying eukaryotic cells. An exemplary technique is detailed in the Examples, below. In general, freeze drying comprises exposing the cells to temperatures below 0° C. while applying a vacuum, and allowing the process of sublimation to remove all or essentially all of the water originally present in the platelets and their surroundings. The resulting platelets are in a solid (dry) form, and can be used in the methods of the invention, below, directly, or after rehydration.

The methods of making a composition may further comprise rehydrating (or reconstituting) the freeze-dried platelets. Rehydrating can comprise adding water or an aqueous solution to the freeze-dried platelets in an amount sufficient to restore at least one physical or biological property to the platelets. Rehydrating can be through any suitable method known in the art, including, but not limited to, direct addition of liquid water to the platelets, and slow vapor reconstitution. Aqueous solutions may comprise any substances that are compatible with platelet function in the amounts in which they are present in the compositions.

The methods of making a composition of the invention can further comprise combining the freeze-dried platelets with other platelets, to form a mixture. The other platelets may be freeze-dried platelets, or may be platelets that are present in a liquid composition, such as blood or a blood fraction (e.g., blood plasma). The mixture is typically, but not always, made in a reaction vessel in which clotting can be detected. That is, although it is possible to make the mixture in vivo by injecting the freeze-dried platelets into a body, typically, the freeze-dried platelets are combined with the other platelets outside of a body, such as in a reaction vessel suitable for detection of blood clots.

The method may further comprise adding one or more substances that have biological activity. For example, the method may comprise adding to a composition comprising the freeze-dried platelets one or more drug or other substance, which may have anti-platelet activity. Exemplary drugs and substances with anti-platelet activities are discussed above.

The method may further comprise adding one or more biological molecules that have enzymatic activity. For example, the method may comprise adding to a composition comprising the freeze-dried platelets one or more coagulation proteins or other substance, which may attenuate platelet activity. Exemplary drugs and substances which attenuate platelet activities are discussed above.

The method may further comprise adding one or more fluorescence molecules to the freeze-dried platelets. For example, the method may comprise adding to a composition comprising the freeze-dried platelets one or more fluorescein or other fluorescence substance, which may enhance the signaling of platelet activity.

In another aspect, the invention provides a method of monitoring one or more functions of platelets. In general, the method comprises obtaining freeze-dried platelets, exposing them to one or more substance that can have an effect on platelet function, and determining whether the substance affected one or more function of the platelets. The method can further comprise reconstituting the freeze-dried platelets before, during, or after exposing them to the substance(s). Obtaining freeze-dried platelets and reconstituting them can be achieved by any of the methods discussed above or known in the art as suitable for such purposes.

Determining the effect of the substance(s) on platelet function can be by any of a wide range of techniques known to those of skill in the art. Such techniques are well known to those of skill in the art, and thus need not be detailed here. Exemplary techniques for determining the effect of the substance(s) on the platelets include, but are not limited to, techniques that assay the ability of the platelets to participate in clot formation (also referred to herein as aggregation when in an in vitro assay). Aggregation can be determined by the amount of light scattering by a composition, and can be determined using a simple photovoltaic cell or a dedicated aggregometer. Molecules that can be used to detect aggregation include, but are not limited to, epinephrine, ADP, thrombin, Thrombin Receptor Activating Peptide (TRAP), collagen, and thromboxane.

Determining the effect of the substance(s) on platelet function can comprise detecting the amount of aggregation of platelet-containing compositions that comprise both freeze-dried platelets and fresh platelets. As discussed in more detail below, the freeze-dried platelets of the present invention have many, if not all, of the functional characteristics of fresh platelets. However, many of the functions are present at levels that are insufficient to promote clotting. Interestingly, although such functions may be at levels insufficient to promote normal levels of clotting, the freeze-dried platelets can participate in normal or near normal clotting if other platelets are present that can provide the insufficient function. Thus, in embodiments, the fresh platelets provide one or more functions that are insufficient or lacking in the freeze-dried platelets, and detection of clotting is possible.

The fact that, in embodiments of the invention, the freeze-dried platelets of the invention have a reduced ability to clot without the aid of other platelets, such as fresh platelets, provides an advantage not provided by fresh platelets alone. In effect, this characteristic makes the freeze-dried platelets, and combinations of freeze-dried platelets and fresh platelets, more sensitive to inhibitors of the clotting system and sensitive to defects in the clotting system. Thus, by use of freeze-dried platelets, one may assay for defects in the clotting system. The assays of the system, and particularly the freeze-dried platelets, allow users to modulate the clotting system of a test sample and make a system that is highly sensitive to small changes in coagulation ability.

Furthermore, combining a pre-determined amount of freeze-dried platelets taken from a donor prior to therapy that affects platelet function with a pre-determined amount of fresh platelets taken from the donor taken after commencement of the therapy (e.g., during or after cessation of the therapy) will create a composition having clotting properties that are equal to or greater than the fresh platelets alone. In effect, this makes the combination of freeze-dried and fresh platelets more sensitive to inhibitors of the clotting system, and makes the combination sensitive to defects in the clotting system. Thus, by use of a composition comprising both freeze-dried platelets and fresh platelets, one may assay for defects in the clotting system with more sensitivity than with fresh platelets alone.

In embodiments of the invention, the freeze-dried platelets preserve the surface markers of fresh platelets. In effect, this makes the platelets sensitive to defects in Glycoprotein IIb/IIIa, Glycoprotein Ib, von Willebrand Factor, and fibrinogen, among other defects. It also makes the platelets more sensitive to Afibrinogenemia, Thromlasthenia, vWF disease, Bernard Souleir Syndrome, Receptor Defects Deisorders of Secretion/signal transduction, Strorage Pool Deficiency, Diminished Thromboxane Synthesis, Signal Transduction/Primary Secretion Defects, and Deficiency of Platelet Coagulant Activities. Thus, by use of freeze-dried platelets, one may assay for platelets defects and defects in the clotting system.

The method of monitoring can comprise obtaining multiple samples from one donor and comparing the samples to each other and/or to a standard curve, to determine the presence and/or level of function of one or more platelet functions. The samples may be obtained over time, and the comparison made to determine the effect of one or more treatment regimens on platelet function or the clotting system in general. They also may be analyzed to confirm that there are adequate numbers of platelets in the donor's blood to support surgery or other procedures where blood might be lost. The samples that are obtained may be stored for short periods of time as fresh samples, or the samples may be processed to create freeze-dried platelet samples, which are later reconstituted and assayed.

Furthermore, the monitoring can comprise obtaining multiple samples from one donor and comparing the samples to each other and/or to a standard curve, to determine the presence and/or level of function of one or more platelet functions. For example, one can assay for Afibrinogenemia, Thromlasthenia, vWF disease, Bernard Souleir Syndrome, Receptor Defects Deisorders of Secretion/signal transduction, Strorage Pool Deficiency, Diminished Thromboxane Synthesis, Signal Transduction/Primary Secretion Defects, or Deficiency of Platelet Coagulant Activities.

In yet another aspect, the present invention provides a method of diagnosing a disease or disorder of the blood clotting system. In general, the method comprises combining freeze-dried platelets with fresh platelets or plasma, and determining whether the mixture has normal levels of one or more function of the blood clotting system, an abnormal level (be it high or low) indicating a disease or disorder.

The freeze-dried platelets may be obtained from one or more donors with a known status with respect to the clotting system (e.g., having a fully functional clotting system, or having a defect in one or more clotting factors). When the freeze-dried platelets are obtained from mixtures of platelets from a public blood bank, they can be assumed to be “normal” or “fully functional” with regard to platelet function. Alternatively, the freeze-dried platelets may be obtained from a patient undergoing or about to undergo a treatment regimen that might affect platelet function. Likewise, the freeze-dried platelets can be obtained from a patient who has completed a treatment regimen that has, or might have, affected platelet function (whether the patient had completed the full treatment regimen or was removed from the regimen early due to adverse side-effects).

Like the freeze-dried platelets, the fresh platelets or plasma may be obtained from one or more donors with a known status with respect to the clotting system (e.g., having a fully functional clotting system, or having a defect in one or more clotting factors). When the fresh platelets or plasma are obtained from mixtures from a public blood bank, they can be assumed to be “normal” or “fully functional” with regard to platelet function or plasma complement. Alternatively, the fresh platelets or plasma may be obtained from a patient undergoing or about to undergo a treatment regimen that might affect platelet function. Likewise, the fresh platelets or plasma can be obtained from a patient who has completed a treatment regimen that has, or might have, affected platelet function (whether the patient had completed the full treatment regimen or was removed from the regimen early due to adverse side-effects).

Regardless of the source of the freeze-dried platelets and the fresh platelets or plasma, the method comprises combining the two to make a mixture. The mixture is then assayed for one or more biological or biochemical functions of the mixture. Preferably, one or more functions of the clotting system, such as the ability to aggregate, are assayed. Comparison of the level of function or activity of the chosen functions or activities to “normal” levels permits one to determine if there is a difference in the levels. A difference in the levels indicates the presence of a disease or disorder of the blood clotting system.

In exemplary embodiments, the method comprises combining freeze-dried platelets that were obtained from a public blood bank with fresh platelets that were removed from a patient having, or suspected of having, a disease or disorder of the blood clotting system to form a mixture, and determining whether the person has a defect in the blood clotting system by assaying one or more biological or biochemical functions of the mixture. According to this aspect of the invention, the defect, if present, decreases or abolishes the patient's blood clotting system's ability to function normally or to cause clot formation in a pre-defined period of time.

In other exemplary embodiments, the method comprises combining freeze-dried platelets obtained from a patient prior to initiation of a treatment regimen with fresh platelets or plasma obtained from the patient at one or more times during or after completion of the treatment regimen to form a mixture. The method further comprises determining the clotting ability of the mixture(s), the ability indicating whether the treatment regimen induced a disease or disorder of the clotting system, or exacerbated an underlying, but never recognized, disease or disorder of the clotting system of the patient.

The freeze-dried platelets and platelets from the patient may be provided from any source, in accordance with the discussion above. Combining of the two can be by any suitable method, such as those well known in the art for combining two eukaryotic cells. Furthermore, determining whether the patient has one or more defect in the blood clotting system can be accomplished by any suitable technique, as discussed above.

In embodiments, determining comprises detecting the presence or amount of aggregation of platelets in the mixture. In general, low levels of aggregation indicate a defect or deficiency in blood clotting activity, whereas high levels of aggregation indicate normal or acceptable levels of activity. Typically, determining whether the patient's blood clotting system is defective comprises assaying clotting time of the mixture.

The method can comprise other steps in addition to the basic steps disclosed above. For example, the method can comprise obtaining freeze-dried platelets prior to combining them with blood. In embodiments, the freeze-dried platelets are obtained from the patient for whom the assay is being performed, and are platelets that were obtained at an earlier time, such as prior to initiation of a drug regimen. The method can also comprise adding one or more drugs or other substances, which have a known effect on platelets or other participating cells or molecules of the clotting system, to the platelets, and determining the effect of the addition on clotting function. By selecting specific drugs with known activities, it is possible to determine the precise cause of the disease or disorder. With such knowledge, appropriate treatment regimens may be implemented.

In a further aspect, the invention provides a method of monitoring the progression of a disease or disorder of the blood clotting system. The method generally comprises combining freeze-dried platelets with fresh platelets or plasma two or more times (for either or both of the freeze-dried platelets, the fresh platelets, and/or the plasma), and determining if a disease or disorder of the blood clotting system is present in the person from whom the freeze-dried platelets or fresh platelets or plasma are obtained. By comparison of two time points, one can determine if a change in the status of the disease or disorder (if present) has occurred between the two time points. This information can, among other things, aid a doctor or patient in deciding whether to continue a particular treatment regimen. Furthermore, typically, multiple assays are performed over time to give an indication of progression of the disease or disorder over time.

In the methods of the invention, determining the presence or absence of a disease or disorder state of the blood clotting system, or progression of a disease or disorder of the blood clotting system generally comprises combining the freeze-dried platelets with fresh platelets or plasma to make a mixture, and determining the blood clotting ability of the mixture. Typically, determining the blood clotting ability of the mixture indicates the blood clotting ability of the patient's blood, and comprises assaying clotting time of the mixture.

As with other methods of the invention, various drugs or other substances can be added to the assay mixture to determine the specific defect in the disease or disorder. Knowledge of the specific source of the defect may enable treatment regimens to be developed.

In still another aspect, the invention provides methods of monitoring the effects of a treatment regimen for a patient on the blood clotting system of that patient. In general, the methods comprise combining freeze-dried platelets and fresh platelets two or more times (for either or both of the freeze-dried platelets, the fresh platelets, and/or the plasma), and determining if a disease or disorder of the blood clotting system is present in the person from whom the freeze-dried platelets or fresh platelets or plasma are obtained. By comparison of two time points, one can monitor the effects of a treatment regimen on the blood clotting system of that person. In this method, either the freeze-dried platelets, the fresh platelets or plasma, or both, can be obtained from the same person (i.e., the patient). The information obtained by comparison of two or more time points can, among other things, aid a doctor or patient in deciding whether to continue a particular treatment regimen.

In embodiments, the method comprises obtaining freeze-dried platelets from a public source or from a patient prior to initiation of a treatment regimen, obtaining fresh platelets or plasma from a patient or a public source prior to initiation of a treatment regimen, and obtaining fresh platelets or plasma from the patient one or more times during a treatment regimen. The method further comprises determining the blood clotting ability of combinations of freeze-dried and fresh components. Typically, determining the blood clotting ability of the mixture indicates the blood clotting ability of the patient's blood, and comprises assaying clotting time of the mixture. Furthermore, typically, multiple assays are performed over time to give an indication of the effects of the treatment regimen over time.

Numerous treatments for a variety of diseases and disorders are available to the public. Some of these treatments, while effectively treating a particular disease or disorder, result in unintended effects (i.e., side-effects) that diminish or abolish one or more functions of the blood clotting system. Other treatments are specifically designed to promote or inhibit the activity of a patient's blood clotting system. In any event, it is often desirable to monitor the presence and/or concentration of drugs in the blood of a patient, and in particular monitor the effect of those drugs on the patient's blood clotting activity. The present methods permit one to monitor such effects simply and rapidly.

It is to be noted that all of the methods of monitoring and diagnosing can comprise one or more control reactions. The concept of control reactions is well known to those of skill in the art, and numerous types of control reactions can be included in the methods of the present invention to monitor the effectiveness and success of one or more steps in the methods. Among the more common control reactions that can be performed are reactions that involve freeze-dried platelets as the sole source of platelets, reactions that involve fresh platelets as the sole source of platelets, reactions in which one or more known substances (with known effects on platelet function or clotting system function) are exposed to fresh platelets (e.g., a positive control), and reactions in which no substance in addition to platelets is added (e.g., a negative control). Included among the control reactions are reactions that generate a standard curve. Because the methods of the present invention provide repeatable aggregation characteristics when performed with accurately measured amounts of normal freeze-dried platelets and normal serum or blood, standard curves can be generated, and these standard curves can be used as a basis for comparison of test samples for any number of characteristics, including, but not limited to, platelet number/concentration, ability of the platelets to participate in clotting, and presence or absence of functional surface proteins on platelets.

It is also to be noted that, although the methods were disclosed as being suitable for use with freeze-dried platelets and fresh platelets or plasma, freeze-dried platelets can be combined according to the methods of the invention with whole blood, platelets, plasma, purified coagulation proteins, and other components of the blood system. Use of the terms “fresh platelets” and/or “fresh plasma” is to be understood to encompass all other types of fresh blood products. Furthermore, the term “fresh” does not necessarily require a strict time-dependency. Rather, it is used solely to differentiate between freeze-dried platelets and non-freeze-dried substances.

The methods of the invention may also comprise performing the method more than one time on the same sample, under the same conditions. As is known in the art, performing a method on multiple identical samples provides an indication of the reliability and reproducibility of the method. According to the present invention, each step in a method, or only certain steps within the method, can be repeated according to this embodiment of the invention.

As is evident from the above description, all the methods of detection and monitoring can encompass the general concept of determining platelet counts or function levels by assaying clot time. Thus, the methods of the invention can be considered, in embodiments, as methods of determining platelet counts of a sample comprising platelets. Likewise, the methods of the invention can be considered, in embodiments, as methods of determining platelet function of a sample comprising platelets. Typically, platelet function is assayed by the ability to participate in the clotting process.

The freeze-dried platelets of the invention show many characteristics of fresh platelets. Among those characteristics is size—the freeze-dried platelets of the invention are of about the same size as fresh platelets. Thus, the freeze-dried platelets can be used to calibrate instruments for detection and study of platelets. Being freeze-dried, the platelets of the invention are advantageously used for calibrating machinery because calibration can be accomplished at any convenient time, rather than in a small window of opportunity provided by fresh platelets.

In an additional aspect, the invention provides kits for performing the methods of the invention. Typically, the kits of the invention comprise freeze-dried platelets. The kits can also comprise some or all of the other reagents and supplies necessary to perform at least one embodiment of one method of the invention. For example, the kits can comprise one or more drugs that affect the ability of platelets to participate in the clotting system. Thus, the kits can be diagnostic kits, blood clotting monitoring kits, or drug treatment monitoring kits. Often, the kits will comprise some or all of the supplies and reagents to perform one or more control reactions to ensure the kits are performing properly and to provide baseline results against which test samples can be compared.

In its simplest form, a kit according to the invention is a container containing at least one composition according to the invention. Thus, in embodiments, the kit of the invention comprises a container containing freeze-dried platelets. In other embodiments, the kit comprises multiple containers, each of which may contain freeze-dried platelets or other substances that are useful for performing one or more embodiment of a method of the invention. In other embodiments, the kit comprises additional components, which may be contained in the same or one or more different containers. Like the compositions it holds, in its various forms, the kit of the invention can comprise substances that are useful for detection and/or study of platelet function; for calibrating instruments; for calibrating platelet size; for calibrating differential gradient separation techniques; as research tools to examine the interaction of platelet receptors and their ligands; to study surface mediated enzymatic reactions, including but not limited to tenase complex, prothrombinase complex, and the like; to study platelet aggregation, whether mechanical or biochemically induced; to study platelet biology and storage; to isolate platelet-related surface molecules; to isolate and purify platelet cytoplasmic molecules or platelet granules (alpha and dense granules); to study platelet and microparticle interactions; to study anti-platelet medications; to study platelet inhibitors; to determine platelet inhibitors that can be tailored to individuals; to study neuropsychopharmacology; to study inflammation, coagulation, cellular repair, and regeneration; to study neo-antigenicity in platelet therapies; to characterize non-MHC antigens that promote immune responses against blood cells; to study the effect of blood-borne pathogens; to image normal and damaged blood vessels; and to study angiogenesis, atherosclerosis, thrombosis, and cardiovascular disease.

The container can be any material suitable for containing a composition of the invention or another substance useful in performing a method of the invention. Thus, the container may be a vial or ampule. It can be fabricated from any suitable material, such as glass, plastic, metal, or paper or a paper product. In embodiments, it is a glass or plastic ampule or vial that can be sealed, such as by a stopper, a stopper and crimp seal, or a plastic or metal cap. In general, the container and seal are made of materials that can be sterilized by heat (dry or wet), radiation (UV, gamma, etc.), or exposure to chemicals. Preferably, the container is sterilized before the composition of the invention is introduced into the container. Typically, the container will be of sufficient size to contain the composition of the invention, yet have head space to permit addition of additional substances, such as sterile water or saline or a mixture of the two, which can be used to rehydrate the composition in the container. In embodiments, the container comprises a sufficient amount of platelet-containing material to perform at least one assay of one embodiment of the method according to the invention. The amount of platelet-containing material contained in the container can be selected by one of skill in the art without undue experimentation based on numerous parameters that are relevant to performing an assay according to the invention (including optional control reactions and repeating of the assay) and packaging and storing of the kit.

In embodiments, the container is provided as a component of a larger unit that typically comprises packaging materials (referred to below as a kit for simplicity purposes). The kit of the invention can include suitable packaging and, optionally, instructions and/or other information relating to use of the compositions. Typically, the kit is fabricated from a sturdy material, such as cardboard or plastic, and can contain the instructions or other information printed directly on it. In embodiments, the kit comprises other components, such as, but not limited to, purified components of the clotting cascade and drugs affecting the clotting cascade. The kit can comprise multiple containers containing the composition of the invention. In such kits, each container can be the same size, and contain the same amount of composition, as each other container, or different containers may be different sizes and/or contain different amounts of compositions or compositions having different constituents. One of skill in the art will immediately appreciate that numerous different configurations of container sizes and contents are envisioned by this invention, and thus not all permutations need be specifically recited herein.

In general, the kit comprises containers to contain the components of the kit, and is considered a single package comprising a combination of containers. Thus, the components are said to be in packaged combination within the kit. In addition to a container containing the composition of the invention, the kit can comprise additional containers containing additional compositions of the invention. Each container may contain enough platelets for a single performance of an embodiment of the method of the invention, or it may contain enough for two or more performances. The various containers may contain differing amounts of the composition of the invention. Thus, in embodiments, the kit comprises a sufficient amount of platelets to perform and embodiment of the method according to the invention. In embodiments, the kit comprises other components, such as purified components of the clotting cascade. The kit can further comprise some or all of the supplies and materials needed to prepare for and perform a method of the invention, such as, but not limited to, sterile water or a sterile aqueous solution (e.g., saline). In some embodiments, the kits comprise one or more liquids to hydrate the compositions of the kits. The liquid may be any suitable liquid, but is typically a water-based liquid, such as water or saline.

### EXAMPLES

Certain features of embodiments of the invention will be further explained by the following examples, which are intended to be purely exemplary of the invention, and should not be considered as limiting the invention in any way.

### Example 1

### Preparation of a Composition of the Invention

For some experiments, platelets were purchased from BRT Labs (Baltimore, Md.) and used either within 4-24 hours of draw or at 6-7 days post draw. For other experiments, fresh platelets were collected into acid citrate dextrose (ACD) anticoagulant buffer (1.5 volumes platelets+8.5 volumes blood). Yet, for other experiments, outdated platelets (George Washington University Blood Banks, Washington D.C.) no longer than 5 days outdated were used.

Platelet Rich Plasma (PRP), whether indated or outdated, was obtained by low speed centrifugation (135×g) for 15 minutes to remove red blood cells. The centrifuged PRP (without red blood cells) was acidified to pH 6.5 by adding 1/14 volumes of ACD and then pelleted by centrifuge at 1000×g for 10 min. The platelet-poor plasma was decanted, and the packed cells were drained over a paper towel to remove plasma proteins. Alternatively, residual liquid was removed by aspiration with a plastic transfer pipette. The platelets were resuspended in 1 ml of Cation-Free Loading Buffer (9.5 mM HEPES; 100 mM NaCl; 4.8 mM KCl; 5 mM glucose; 12 mM NaHCO₃; 50 mM trehalose; prepare as 10× concentrate, adjust pH to 7.4 with HCl, then reduce to pH 6.8 with ACD, then dilute to 1× with deionized ultrafiltered water) containing 1% ethanol. The concentration of platelets was adjusted to give about 1.0×10⁹ platelets per ml. The mixture was incubated for 2 hours at 37° C., mixing once every thirty minutes. Finally, the albumin (BSA) concentration was adjusted to 5% (w/v) of the platelet preparation for lyophilization. Then Ficoll-400, concentration adjusted to 6% (w/v) of the platelet preparation, was added as a bulking reagent excipient prior to lyophilization.

The lyophilization was performed as described in Table 1 or Table 2, using the Advantage Wizard 2.0 lyophilizer from Virtis, Inc., Warminster, Pa.):

TABLE 1
Lyophilization Protocol

| Temperature (° C.) | Time (minutes) | Vacuum (mTorr) |
|---|---|---|
| −45 | 120 | none |
| −40 | 150 | max (about 100) |
| −20 | 100 | max (about 100) |
| −10 | 100 | max (about 100) |
| 0 | 100 | max (about 100) |
| 10 | 100 | max (about 100) |
| 20 | 100 | max (about 100) |
| 25 | 100 | max (about 100) |
| | (or longer) | |

* condenser was set to −65° C.

TABLE 2
Lyophilization Protocol

Shelf Temp (° C.)

| Period | Time (h) | Start | End | Vacuum (mTorr) |
|---|---|---|---|---|
| 1 | 0.63 | 30 | −45 | ambient |
| 2 | 4 | −45 | −45 | ambient |
| 3 | 1 | −45 | −40 | 100 |
| 4 | 12 | −40 | 30 | 100 |
| 5 | 12 | 30 | 30 | 100 |

Freeze-dried platelets were heat fixed by exposing the composition to 75-80° C. for 24 hrs. In certain preparations, the lyophilized platelets were irradiated to sterilize them. Irradiation was performed using standard protocols at 15, 30, and 50 kGy.

### Example 2

### Evaluation of the Physical Characteristics of a Composition

The structural composition of a composition prepared according to Example 1 was examined using the Beckman Multisizer 3 COULTER COUNTER (Fullerton, Calif.), particularly to analyze particle size. The multisizer provides size and volume distributions with a range up to 10 um. As used herein, the volume of a platelet is 2-4 um where as anything less than 1 um is considered to be platelet microparticles.

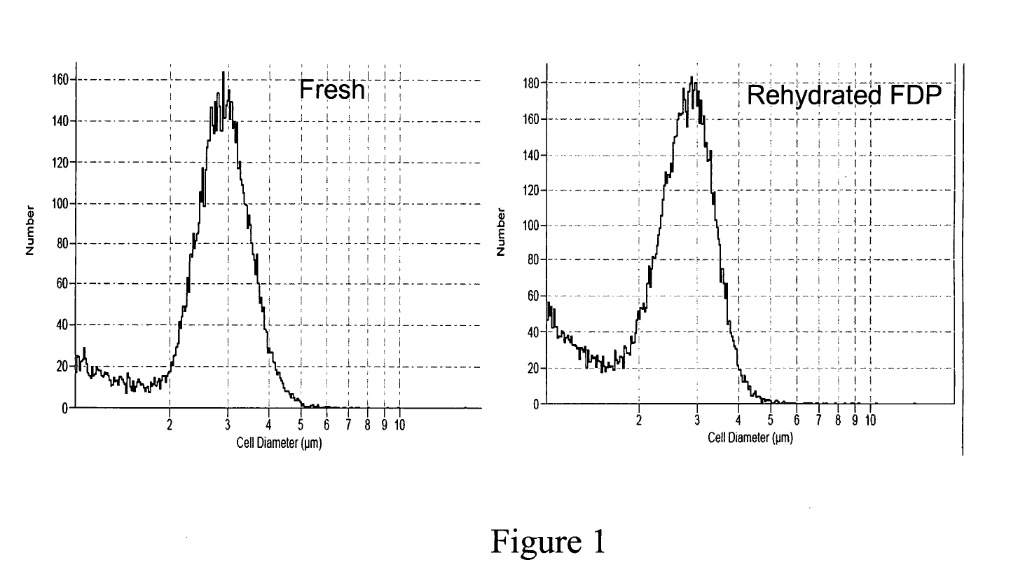

It is clear from the data presented in Examples 1 and 2 that a composition of the invention, upon reconstitution with water, retained a size similar to fresh platelets. Furthermore, as can be seen from FIG. 1, the protocol for preparing freeze-dried platelets can result in a composition comprising mostly platelets and, to a small extent, some microparticles. More specifically, FIG. 1 depicts the results of analyses of size ranges of compositions prepared according to the method disclosed in Example 1. Upon rehydration, the rehydrated particles showed a mixture of platelets and platelets microparticles, as evidenced by the sizing data (FIG. 1). It is estimated that the percentage of microparticles is somewhere between about 1-20% of the total number of particles in the composition.

### Example 3

### Use of Freeze-Dried Platelets as Calibrating Reagents for Normal Pooled Plasma

As discussed above, it has been found that freeze-dried platelets can be used to monitor functions of platelets. In this vein, the ability of freeze-dried platelets to participate in blood clotting was determined. To do so, various amounts of freeze-dried platelets were mixed with plasma pooled from numerous normal donors, and the time required to generate a clot was determined.

To assay clotting time, 100 ul of APCT (activated plasma clot time, Analytical Control Systems, Inc., Fishers, Ind.) reagent was mixed with 25 ul of various concentrations of water-reconstituted freeze-dried platelets and 25 ul of normal pooled plasma obtained from commercial suppliers. The mixture was incubated at 37° C. in a water bath for 3 minutes, then 100 ul of 0.02 M CaCl₂ (37° C.) was added, and clot time determined.

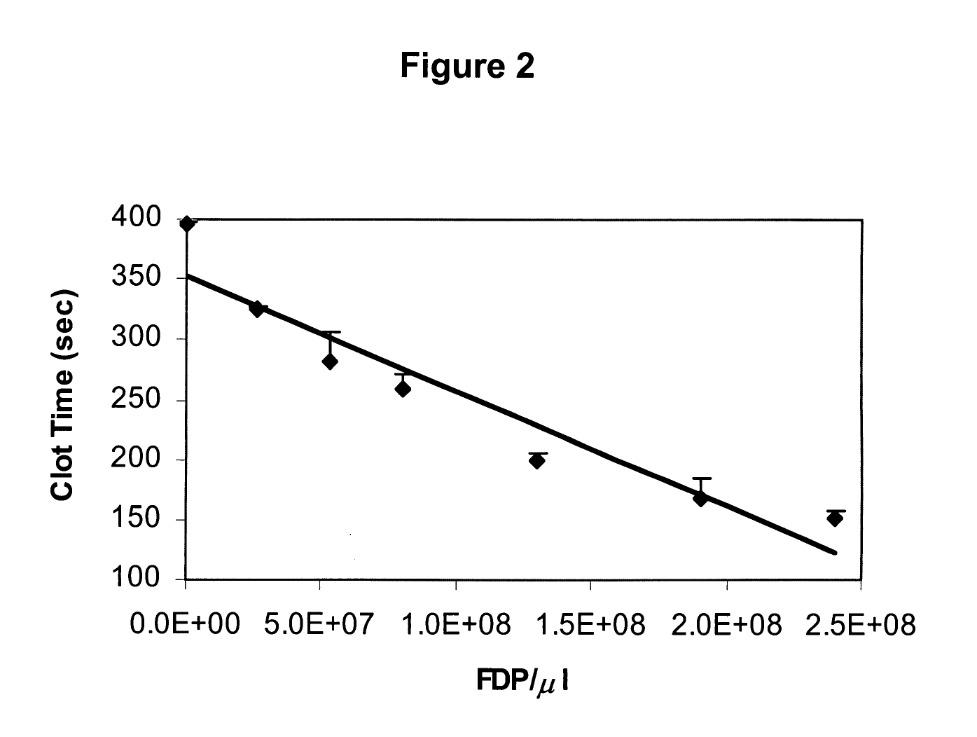

As can be seen from FIG. 2, the amount of freeze-dried platelets added to a given amount of normal plasma yields a standard curve in which clot time is proportional to the amount of freeze-dried platelets. Thus, the freeze-dried platelets can not only participate in clot formation, but can be used to identify normal clotting times for plasma. By comparison to the normal time for clotting for a given amount of freeze-dried platelets and plasma, one can identify anomalies in the blood clotting abilities of individual samples, such as those obtained from patients having or suspected of having a disease or disorder of the blood clotting system.

A standard clotting assay relies on platelet factor 3 (phospholipid) to activate the intrinsic coagulation mechanism. Other assays use fresh platelets to supply the phospholipid component. In the present invention, the phospholipid is supplied by freeze-dried platelets rather than fresh platelets. Thus, the experiments show not only that freeze-dried platelets have similar physical properties as fresh platelets, but that they have similar functionalities as well.

### Example 4

### Use of Freeze-Dried Platelets as Calibrating Reagents for Platelet Poor Plasma

The concept of the ability of freeze-dried platelets to give standard clotting time responses when mixed with normal plasma was extended to determine if freeze-dried platelets could serve as a calibrating agent for platelet-poor plasma. That is, previous experiments proved that freeze-dried platelets could participate, in a reproducible and predictable way, in blood clotting in mixtures containing normal plasma. Experiments were performed to determine whether freeze-dried platelets could likewise participate in clotting reactions in conjunction with plasma that was abnormal in that it was deficient in platelets. Platelets were purposely removed from the plasma, and freeze-dried platelets were added in order to replace the fresh platelets. The count of fresh platelets in the sample was negligible (about 5000 platelets/ul).

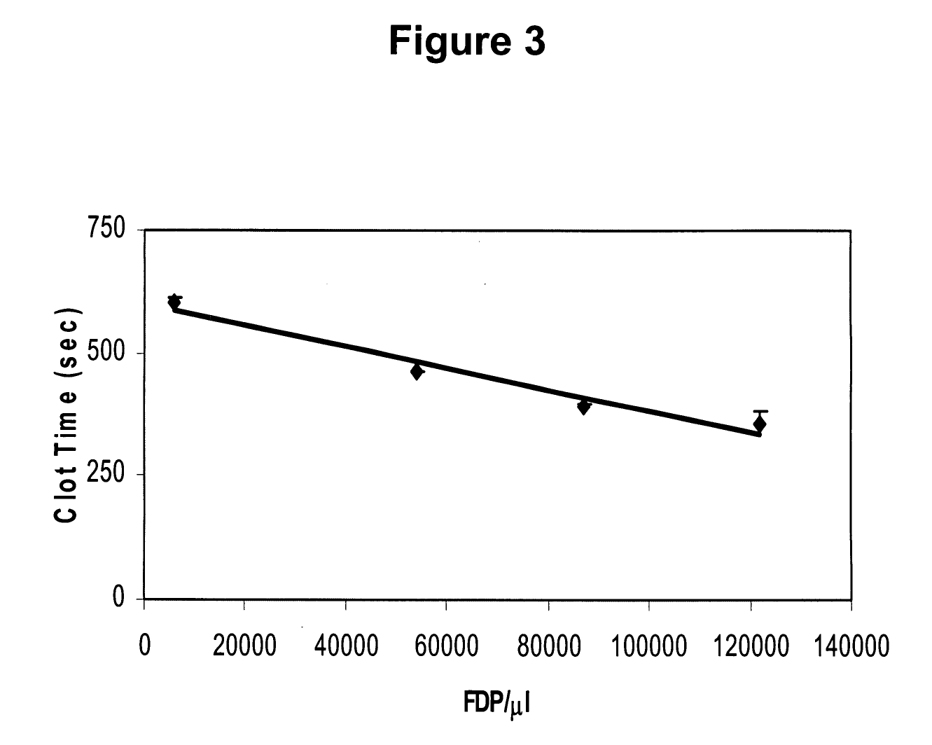

As can be seen from FIG. 3, the amount of freeze-dried platelets added to a given amount of platelet-poor plasma yields a standard curve in which clot time is proportional to the amount of freeze-dried platelets. Thus, the freeze-dried platelets can not only participate in clot formation in platelet-poor plasma, but can be used to identify clotting times for such plasma. By comparison to the normal time for clotting for a given amount of freeze-dried platelets and normal plasma, one can not only identify anomalies in the blood clotting abilities of individual samples, such as those obtained from patients having or suspected of having a disease or disorder of the blood clotting system, but one can also quantitate the number of platelets in the platelet-poor sample. Indeed, one conclusion that can be drawn from this experiment is that, in plasma without any platelets (or plasma with extremely low platelet counts), freeze-dried platelets can be used as a calibrating agent to calibrate for other blood components (i.e., coagulation factor inhibitors or any other defect within the coagulation pathways). In normal plasma, freeze-dried platelets can also be used as a calibrating agent for the same purpose. The system disclosed here uses freeze-dried platelets as a reagent in any given plasma samples independent of platelets present to probe for coagulation protein defects or to probe for certain coagulation inhibitors. For example, in hemophilia plasma, freeze-dried platelets were used on frozen plasma with various defects and were able to identify and correct factor IX, X, and XI defects, but not factor VIII and II defects. One value in this is that a lab can receive frozen plasma and using this freeze-dried platelet reagent to rapidly determine coagulation protein defects.

This Example shows that, in plasma with out any platelets (or plasma with extremely low platelet counts), freeze-dried platelets can be used as a calibrating agent to calibrate for other blood components (i.e., coagulation factor inhibitors or any other defect within the coagulation pathways). It is evident then that, in normal plasma, freeze-dried platelets can also be used as a calibrating agent for the same purpose. The system can use freeze-dried platelets as a reagent in any given plasma sample, independent of whether platelets are present, to probe for coagulation protein defects or to probe for certain coagulation inhibitors. For example, in the hemophilia plasma, freeze-dried platelets were used with frozen plasma having various defects. The combination was able to identify and correct factor IX, X, and XI defects. Correction of defects in factor VIII and II were not shown, however. One advantage of this system is that a lab can receive frozen plasma and, using the freeze-dried platelets and systems of the present invention, rapidly determine coagulation protein defects.

### Example 5

### Use of Freeze-Dried Platelets as Diagnostic Reagent for Coagulation Factor Defects

With the knowledge that freeze-dried platelets can be used to identify defects in clotting ability of plasma, experiments were designed to determine whether freeze-dried platelets can be used to identify specific defects in the blood clotting pathway. To assay clotting time, 100 ul of APCT (activated plasma clot time, Analytical Control Systems, Inc., Fishers, Ind.) reagent was mixed with 25 ul of various concentrations of water-reconstituted freeze-dried platelets and 25 ul of factor deficient plasma obtained from commercial suppliers. The mixture was incubated at 37° C. in a water bath for 3 minutes, then 100 ul of 0.02 M CaCl₂ (37° C.) was added, and clot time determined.

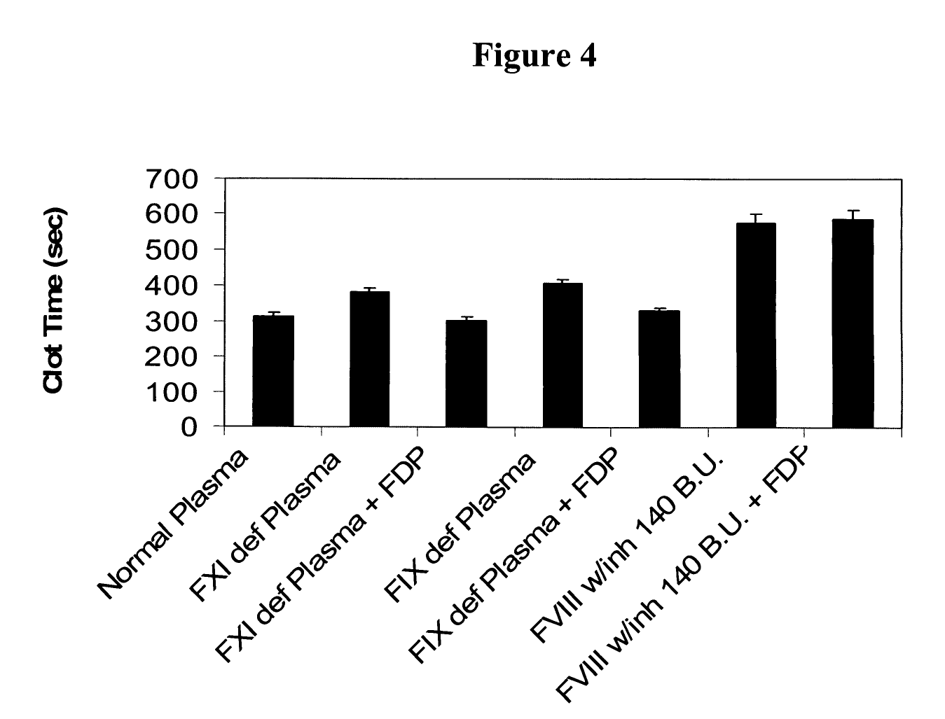

As can be seen in FIG. 4, freeze-dried platelets can overcome the clotting deficiencies of defects in clotting factors XI, X, and IX, but not VIII. Thus, assays can be performed to distinguish between clotting defects based on factor VIII as compared to factors XI, X, and IX, and can identify deficiencies in the intrinsic pathway of clot formation. Because freeze-dried platelets can overcome factor XI, X, and IX defects, a calibration curve can be set up to accurately determine the amount of these factors' presence or absence in blood. By the same token, for patients on warfarin (coumadin), where vitamin-K dependent factors are compromised, freeze-dried platelets can be used to monitor for the deficiency.

### Example 6

### Use of Freeze-Dried Platelets as a Diagnostic Tool to Identify Specific Coagulation Factor Defects

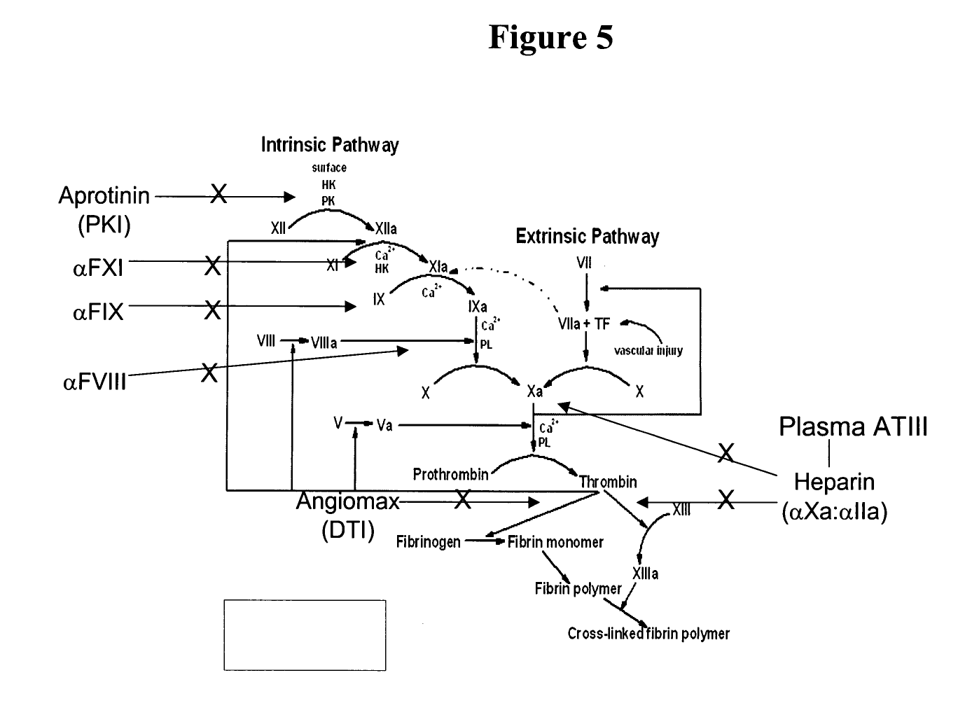

With the realization that freeze-dried platelets can be used to identify defects in intrinsic coagulation factors in a plasma based system, the ability of freeze-dried platelets to be used as a diagnostic tool to pinpoint the same kind of defects in a whole blood system was tested. The ability to do this would distinguish freeze-dried platelet-base diagnostics from other commercially available assays (e.g., aPTT, PT, ELISA, PCR etc.), where whole blood has to be processed to extract plasma, serum, or individual blood components to quantitatively determine the specific defects. For ease of reference, FIG. 5 depicts an overview of the blood coagulation system and blood coagulation inhibitors.

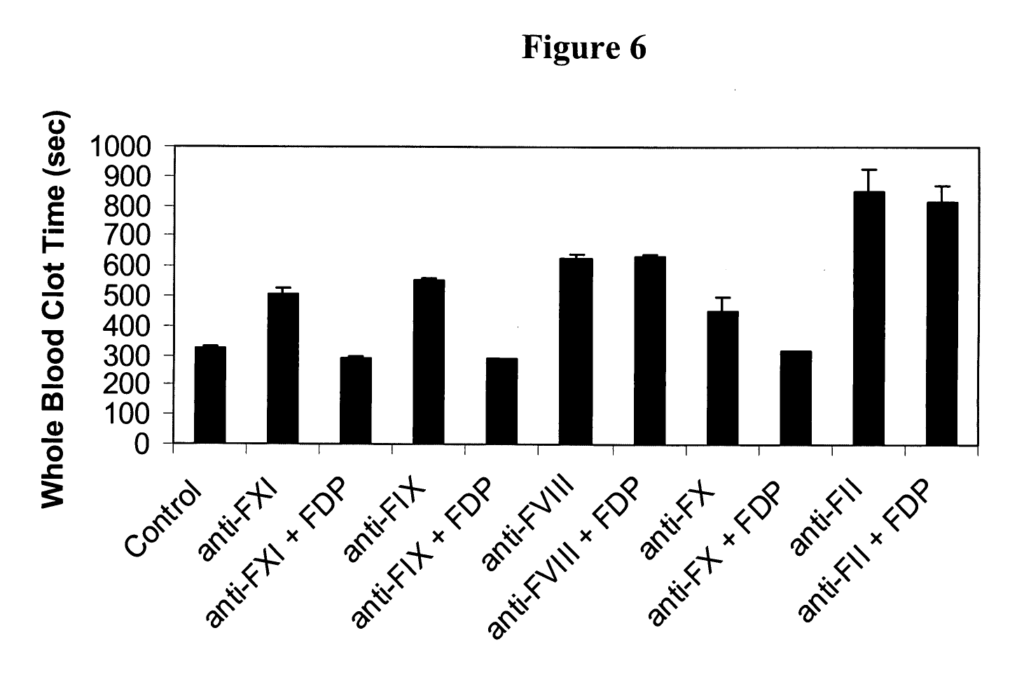

FIG. 6 depicts the effect of freeze-dried platelets on the clotting ability of blood samples having a known defect in a blood clotting component. The data shown in the Figure was obtained as follows: clot time as determined for a mixture containing 400 ul of ACD whole blood (either incubated with various antibodies targeted against specific coagulation factors or with anti-coagulation drugs that are currently used in health care facilities), 25 ul of 0.2 M CaCl₂, 25 ul saline, and 50 ul of various concentrations of reconstituted (rehydrated) freeze-dried platelets.

As can be seen from the Figure, the results of the whole blood assays agree with those of the plasma based assays. Freeze-dried platelets were able to reduce clotting times for defects in factors IX, X, and XI, but not factor VIII. This result indicates that freeze-dried platelets can be used in conjunction with both plasma and whole blood to identify defects in factors IX, X, and XI, and distinguish those defects from those of factor VIII. One advantage of this is that freeze-dried platelets can work well with whole blood, thus avoiding the complication of processing plasmas.

This Example demonstrates that the reaction profiles of freeze-dried platelets are virtually the same as that of the plasma-based system when specific antibodies are added to the whole blood. Furthermore, when whole blood was treated with various anticoagulant drugs, it was found that freeze-dried platelets are also sensitive to these anticoagulants with different kinetics and reaction profiles (see below).

Thus, it has been found that the use of freeze-dried platelets has several distinctive advantages, including:

freeze-dried platelets can be used as a stand alone reagent to identity defects in factors involved in the intrinsic pathway;

freeze-dried platelets can be used with any existing clinical equipment known to be suitable for use with fresh platelets;

freeze-dried platelets can be used in conjunction with exiting diagnostic kits as calibrate reagent; and

freeze-dried platelets can be used with whole blood or plasma to identity defects in factors involved in the intrinsic pathway.

### Example 7

### Freeze-Dried Platelets Show Distinctive Reaction Profiles with Whole Blood

With the knowledge that freeze-dried platelets can be used to identify specific defects in blood clotting systems, the ability of such platelets to identify the presence or effect of various anti-coagulants was tested. Freshly drawn blood in ACD was incubated with the indicated amount of inhibitors. The freeze-dried platelets, at various concentrations, were added and incubated at room temperature for 30 seconds. Blood was then recalcified with 10 mM CaCl₂ and clot time was determined.

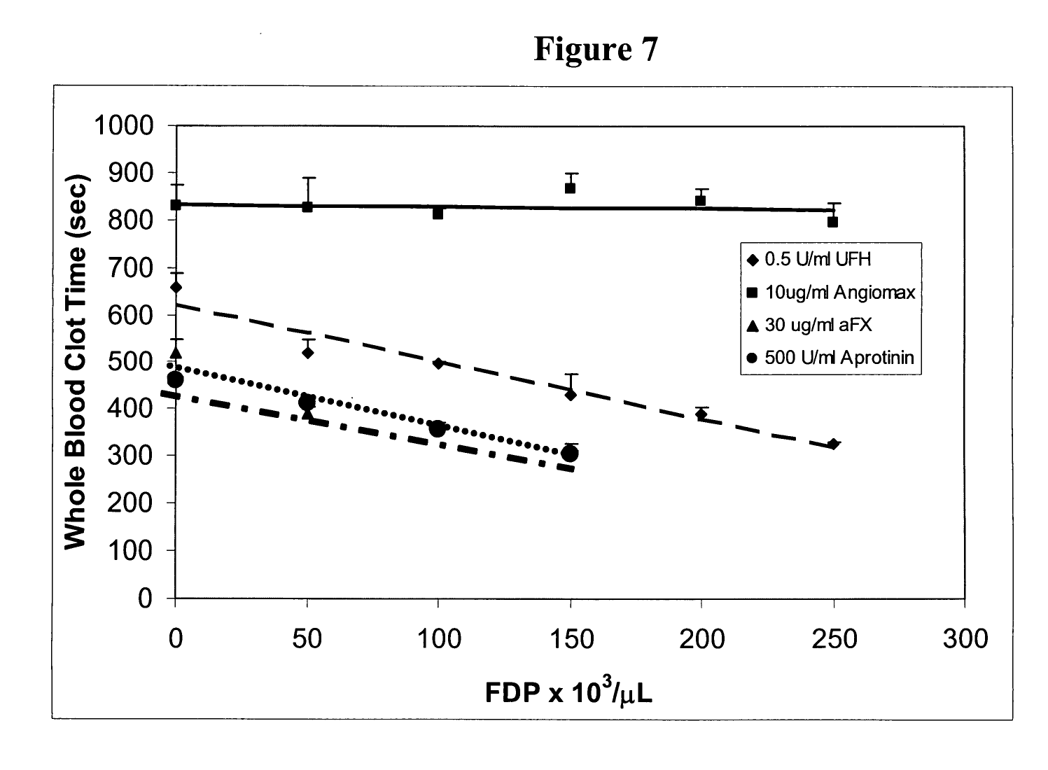

As can be seen from FIG. 7, freeze-dried platelets can be used to identify the presence and/or effect of anti-coagulants in whole blood. Because the freeze-dried platelets react to specific anti-coagulants with distinctive reaction profiles, they can be used not only to detect the presence of the anti-coagulant, but to determine how much of the anti-coagulant is present in the blood. In this way, the anti-coagulant can be monitored in the blood, for example, to ensure that the proper dose is being taken. This is particularly helpful for cardiopulmonary by pass (CBP) patients who are on heparin therapy. Blood from these patients can be monitored at the bedside to determine the levels of heparin in the blood and when it would be safe for surgery.

### Example 8

### Use of Freeze-Dried Platelets to Monitor Vitamin-K Dependent Clotting Factors

Many clotting factors in the clotting cascade are vitamin-K dependent and bind to negatively charged phospholipids on cell membranes. In addition, the Annexin-V marker is a marker for platelet pro-coagulant activity, as it binds to negatively charged phospholipids in a Ca²⁺-dependent manner similar to vitamin-K dependent proteins. To analyze binding of these proteins to freeze-dried platelets, the following experiments were performed on a Becton Dickenson FACS caliber instrument using log-log settings. Platelets were characterized by their representative forward and side scatter light profiles (performed using gel filtered platelets) and/or by the binding of the Fluorescence-labeled proteins. Platelets were diluted to ˜50,000 per ul in HBMT in separate tubes and Fluorescence-labeled proteins were added at saturation for 30 minutes at ambient temperature. Samples were diluted with 2 ml HMBT and 10,000 individual events collected. The fluorescence histogram and percentage of positive cells were recorded, and this represented the platelet population that bound to the fluorescence labeled protein.

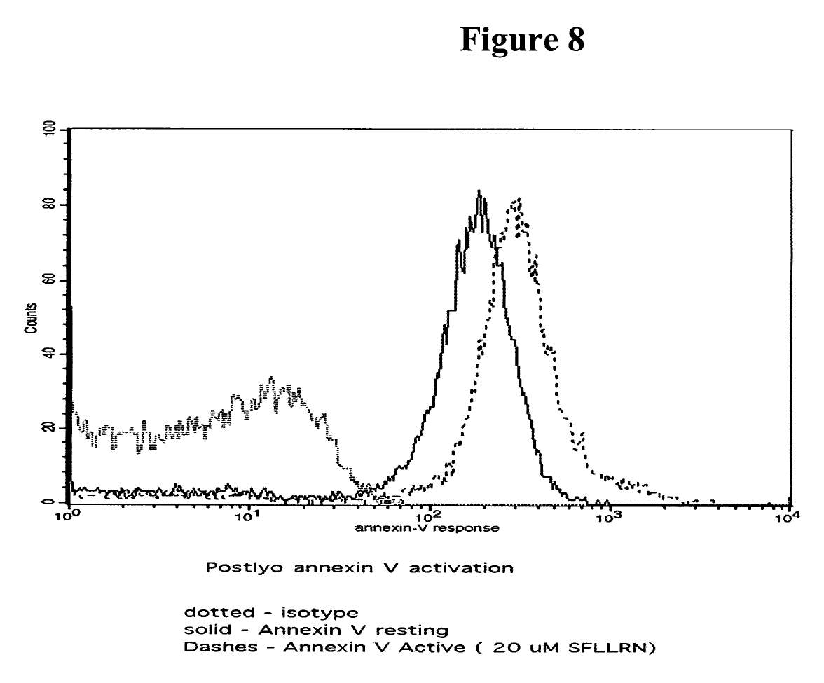

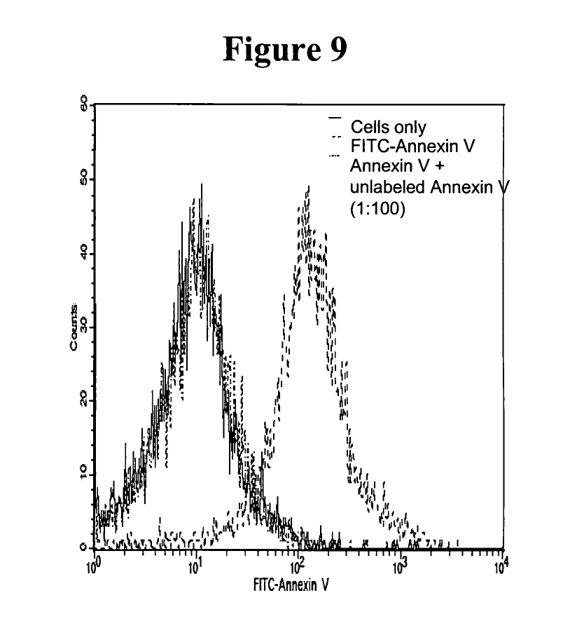

As can be seen from FIG. 8, freeze-dried platelets bind to 25 mM of FITC-labeled Annexin-V (denoted Annexin V resting). Upon addition of with 20 uM of the TRAP peptide (SFLLRN), freeze-dried platelets exposed additional negatively charged phospholipids, resulting in additional Annexin V binding (denoted Annexin V Active). To ascertain that the binding of FITC-Annexin V to resting freeze-dried platelets is specific, 100-fold excess of unlabeled Annexin V was added. As can be seen from FIG. 9, the binding of FITC-Annexin V can be competed off by unlabeled Annexin V, suggesting that the negatively charged surface of the freeze-dried platelets is structured with defined binding sites.

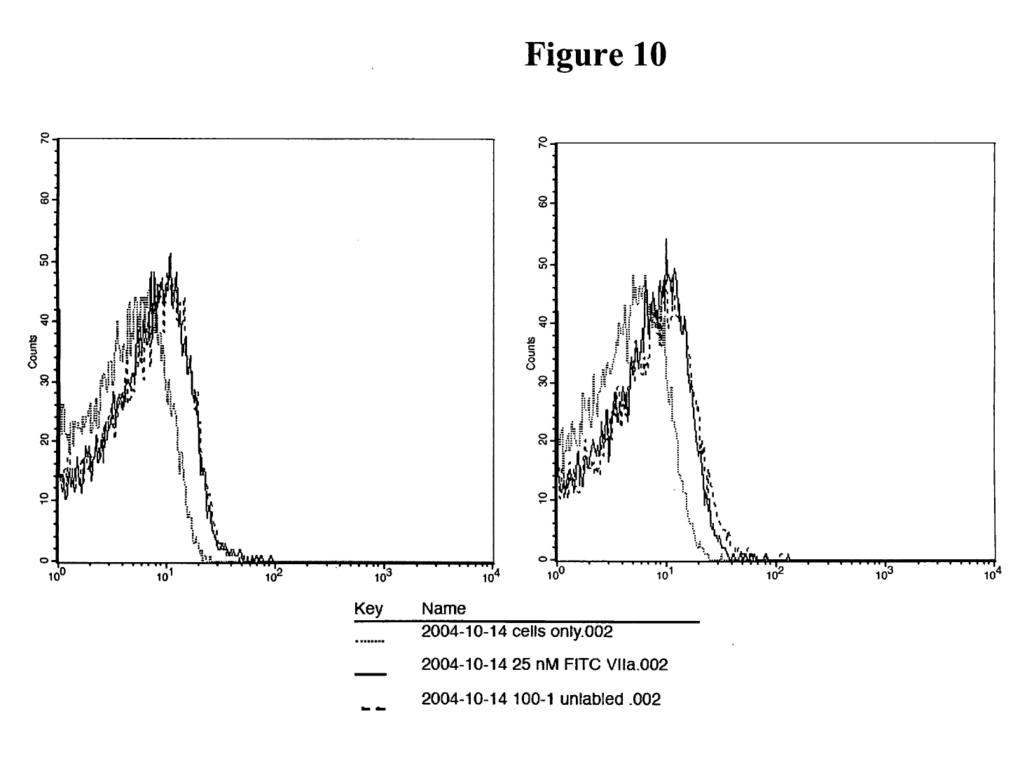

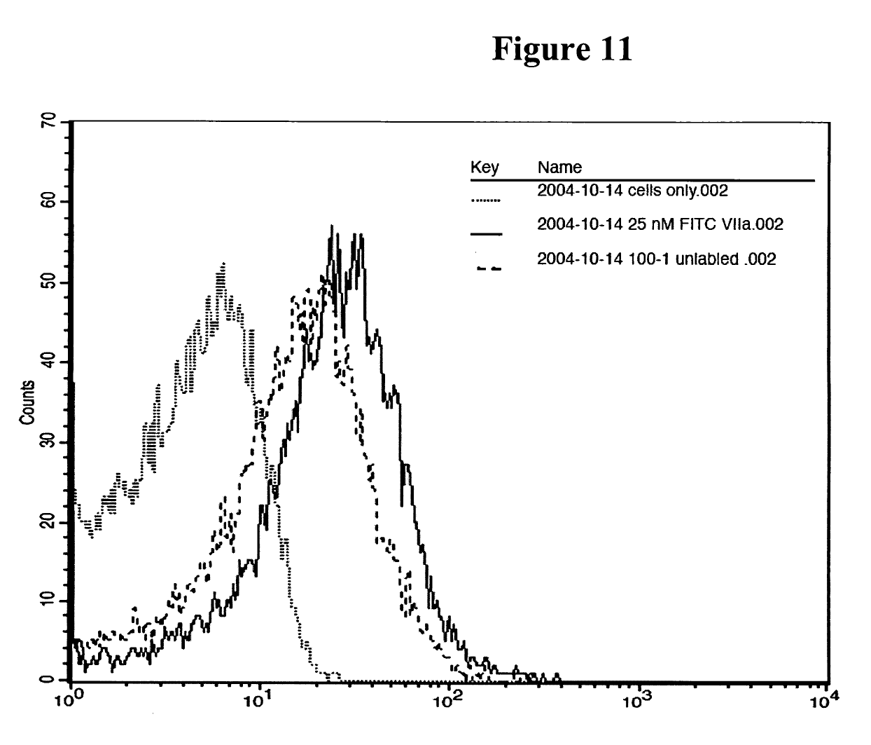

To be more specific, vitamin K dependent proteins were used in the binding assay. When FITC-labeled PPACK-FVIIa (active site inhibited FVIIa) was tested for binding, it was found that FVIIa failed to bind to fresh unactivated platelets as well as fresh activated platelets at a concentration of 25 nM (FIG. 10). However, when freeze-dried platelets were used, FITC-FVIIa showed specific binding at 25 nM and this binding can be competed with unlabeled FVIIa (FIG. 11).

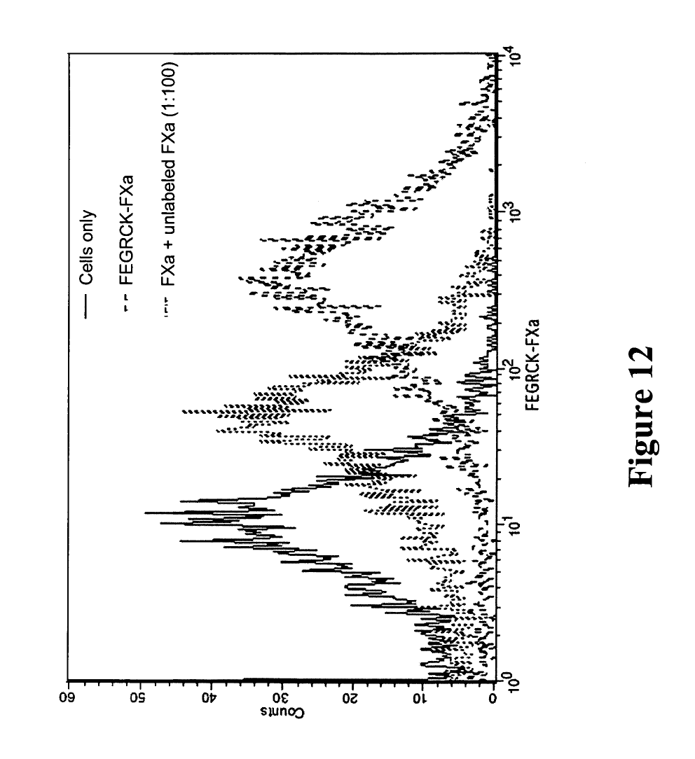

The binding of FITC-labeled EGR-FXa (active sited inhibited FXa) to freeze-dried platelets was also investigated. As can be seen in FIG. 12, the binding of FXa to freeze-dried platelets was specific since it can be competed off by 100-fold excess unlabeled FXa.

Thus, an advantage of using freeze-dried platelets to monitor vitamin K dependent coagulation factors functionality or concentration in whole blood or plasma is evident from these experiments. These coagulation factors bind to the surface to of the freeze dried platelets in a specific manner. Furthermore, this specific binding to the surface of the freeze-dried platelets can be modified. For example, the surface of freeze-dried platelets can be coupled to an agent (luminescence or otherwise) that is specific to each of the Vitamin K dependent factors. The signal (luminescence or otherwise) can be interpreted to pinpoint the identity of the missing factor(s) or factor(s) that is or are under the influence of anticoagulation medication.

### Example 9

### Use of Freeze-Dried Platelets as Diagnostic Reagents to Identify Platelet Defects

Other experiments showed that the freeze-dried platelets of the invention have similar physical and functional characteristics as fresh platelets. To better characterize the physical characteristics, freeze-dried platelets were tested for their response to various agonists that are known to have an inhibitory action on coagulation of fresh platelets.

The experiments in this Example were performed as follows: fresh platelets and/or freeze-dried platelets were diluted to a final concentration of 250,000 platelets per ul in HEPES-Tyrodes Buffer containing 0.3% bovine serum albumin (BSA). Various agonists were added to each composition, as outlined below. 400 ul of the composition was placed in aggregometry cuvettes, and aggregation of the platelets followed over time.

Alpha-FIIa: 0.05-1 U/ml;

Gamma-FII: 0.03 ug/ml;

A23187: 10 mM;

Thrombin Receptor Activating Peptide (TRAP): SFLLRN: 10 mM;

Risto +: 1 mg/ml (20% Autologous Citrated Plasma);

Risto −: 1 mg/ml;

Collagen (Chronolog): 10 ug/ml;

Epinephrine: 300 uM;

Arachidonic Acid: 0.5 mg/ml;

ADP: 20 uM;

Control: No Agonist.

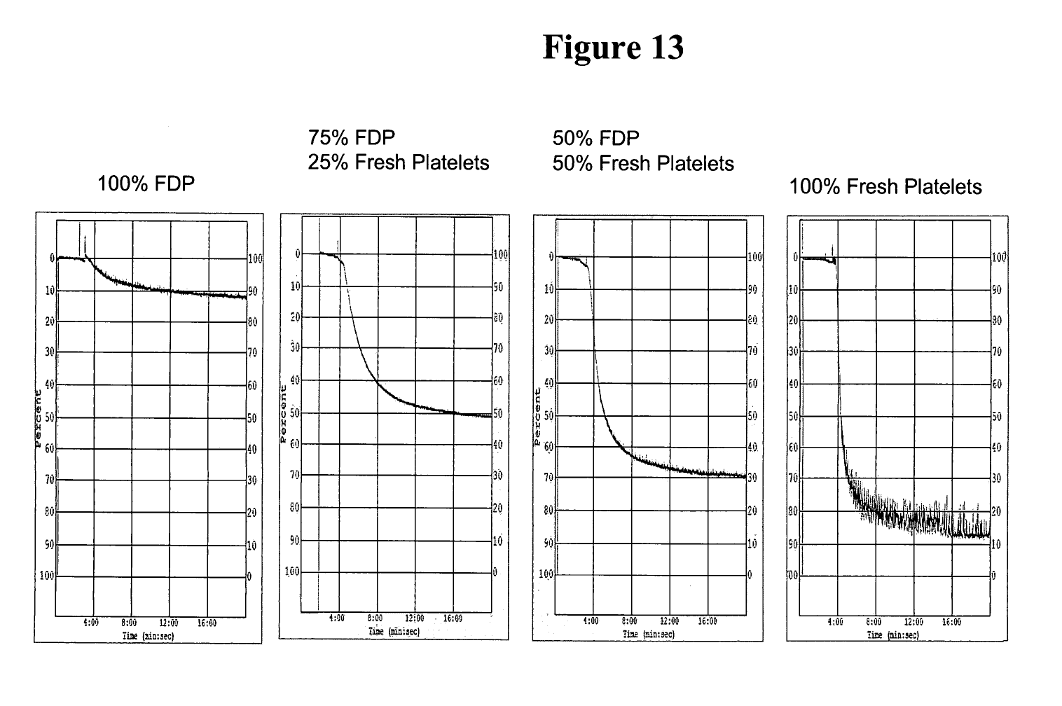

The results of the assays using collagen are presented in FIG. 13. Panel A depicts the percent aggregation when 100% freeze-dried platelets were used. The panel shows a low amount of aggregation (about 10%), indicating that the freeze-dried platelets are only partially sensitive to collagen. In contrast, Panel D shows the effect of collagen on fresh washed platelets. In Panel D, almost 90% aggregation is seen over the same time period that resulted in a little over 10% aggregation of freeze-dried platelets. As can be seen from Panels C and D, mixtures of varying amounts of freeze-dried platelets and fresh platelets gives intermediate levels of aggregation, the amount being dependent on the relative amounts of freeze-dried platelets and fresh platelets added.

In a second set of experiments designed to determine the effect of freeze-dried platelets on the aggregating function of fresh platelets, varying amounts of freeze-dried platelets (rehydrated platelets, or RH) were combined with varying amounts of fresh platelets. The reconstituted platelets were mixed with fresh platelets at the concentrations indicated. To each of these, 10 ug/ml (400 ul platelets+4 ul of 200 mM MgCl (2 mM)+4 ul of 1 mg/ml Collagen (10 ug/ml)) were added to the mixture. After 5 minutes at room temperature, platelets were counted using a standard Complete Blood Count machine (ACT 10 from Beckman coulter).

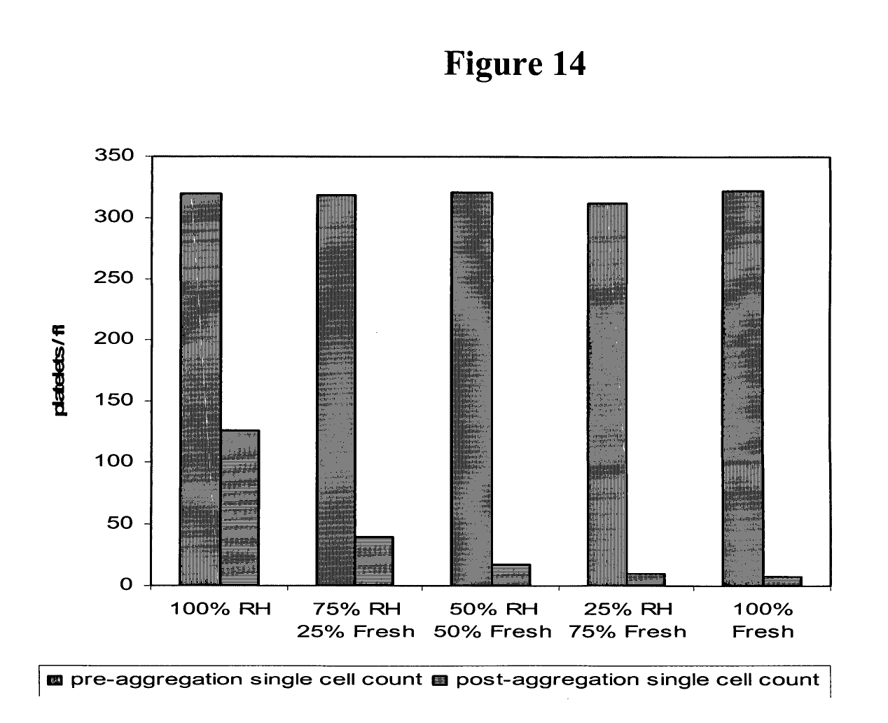

As can be seen from FIG. 14, various mixtures of freeze-dried platelets and fresh platelets have intermediate aggregating characteristics, depending on the relative amounts of each present in the mixture.

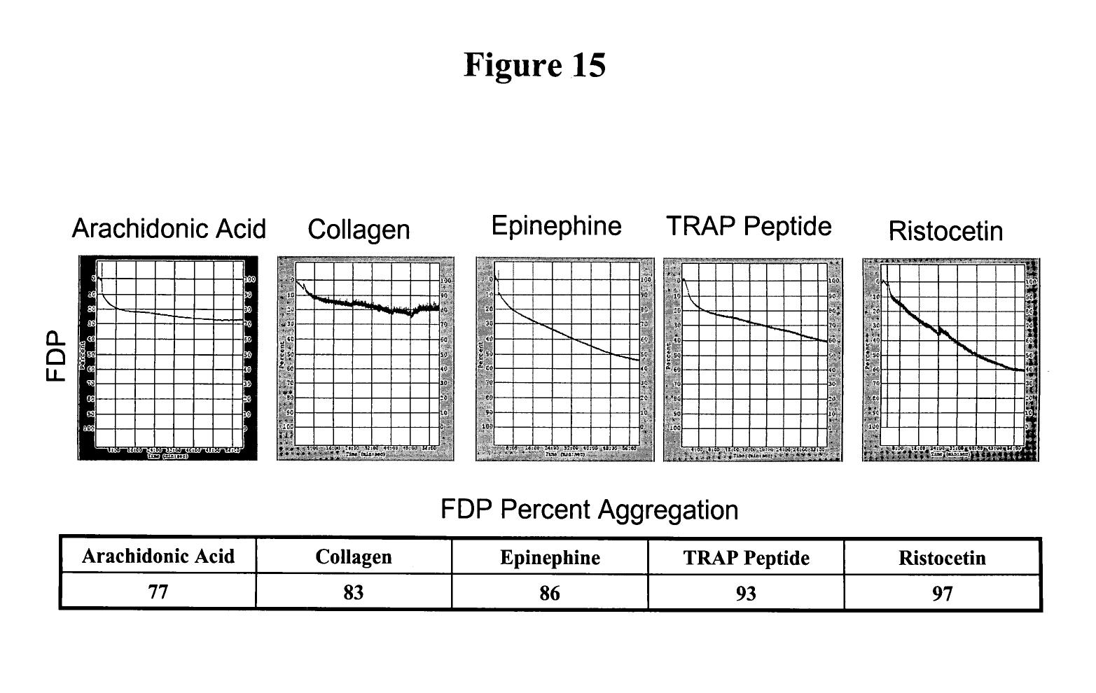

Furthermore, it was also seen that freeze-dried platelets aggregated in response to Arachidonic Acid, Collagen, Epinephrine, thrombin receptor activating peptide (TRAP) and Ristocetin, with aggregation percentage determined to be 77, 83, 86, 93, and 97 respectively (FIG. 15).

The results of FIGS. 13, 14, and 15 indicate that freeze-dried platelets contain at least partially functional receptors that are responsive to all agonist listed above, and have low, but detectable levels of self-aggregation. In a reaction where freeze-dried platelets were mixed with fresh platelets, we demonstrated that the mixture was able to aggregate synergistically in a dose dependant manner. Thus, the use of freeze-dried platelets as a platelet specific diagnostic tool offers several advantages in its various embodiments:

a unique technology to perform such assays—platelets that are fixed with formaldehyde agglutinate do not aggregate, whereas freeze-dried platelets according to the invention are;

freeze-dried platelets preserve relevant surface markers that can be used to monitor platelet function defects, such as Glanzman's thrombasthenia, Bernard-Soulier syndrome, Gray platelet syndrome, Quebec Platelet disorder, Hermansky-Pudlak Syndrome, Chediak-Higashi syndrome, Wiskott-Aldrich syndrome, release defects, vWF disorder, Afibrinogenenia, Scott syndrome, and other congenital disorders;

a patient's own platelets can be freeze-dried and used as a control reagent to monitor the patient's own platelet function during the course of a therapy regimen;

pooled platelets can be freeze-dried and used as global platelet reagent for the same purpose; and

compositions of the invention can be stand-alone products, which can be used on any existing equipment that is suitable for analysis of platelets.

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only, with a true scope and spirit of the invention being indicated by the following claims.

### REFERENCES

Christenson, J. T. and A Kalangos, 2004, Autologous fibrin glue reinforced by platelets in surgery of ascending aorta*: Thorac. Cardiovasc. Surg., v. 52, p. 225-229.

Gilbert, G. E., et al., 1991, Platelet-derived microparticles express high affinity receptors for factor VIII: J. Biol. Chem., v. 266, p. 17261-17268.

Hoffman, M., et al., 1992, Coagulation factor IXa binding to activated platelets and platelet-derived microparticles: a flow cytometric study: Thromb. Haemost., v. 68, p. 74-78.

Holme, P. A., et al., 1995, Platelet-derived microvesicles and activated platelets express factor Xa activity: Blood Coagul. Fibrinolysis, v. 6, p. 302-310.

Mazzucco, L., et al., 2004, The use of autologous platelet gel to treat difficult-to-heal wounds: a pilot study: Transfusion, v. 44, p. 1013-1018.

Nieuwland, R., et al., 1997, Cell-derived microparticles generated in patients during cardiopulmonary bypass are highly procoagulant: Circulation, v. 96, p. 3534-3541.

Oikarinen, K. S., et al., 2003, Augmentation of the narrow traumatized anterior alveolar ridge to facilitate dental implant placement: Dent. Traumatol., v. 19, p. 19-29.

Pierce, G. F., et al., 1989, Platelet-derived growth factor and transforming growth factor-beta enhance tissue repair activities by unique mechanisms: J. Cell Biol., v. 109, p. 429-440.

Prior, J. J., et al., 1999, A sprayable hemostat containing fibrillar collagen, bovine thrombin, and autologous plasma: Ann. Thorac. Surg., v. 68, p. 479-485.

Rosing, J., et al., 1985, Impaired factor X and prothrombin activation associated with decreased phospholipid exposure in platelets from a patient with a bleeding disorder: Blood, v. 65, p. 1557-1561.

Sims, P. J., et al., 1988, Complement proteins C5b-9 cause release of membrane vesicles from the platelet surface that are enriched in the membrane receptor for coagulation factor Va and express prothrombinase activity: J. Biol. Chem., v. 263, p. 18205-18212.

Sims, P. J., et al., 1989, Regulatory control of complement on blood platelets. Modulation of platelet procoagulant responses by a membrane inhibitor of the C5b-9 complex: J. Biol. Chem., v. 264, p. 19228-19235.

Steed, D. L., 1997, The role of growth factors in wound healing: Surg. Clin. North Am., v. 77, p. 575-586.

Tans, G., et al., 1991, Comparison of anticoagulant and procoagulant activities of stimulated platelets and platelet-derived microparticles: Blood, v. 77, p. 2641-2648.

Wajon, P., et al., 2001, Intraoperative plateletpheresis and autologous platelet gel do not reduce chest tube drainage or allogeneic blood transfusion after reoperative coronary artery bypass graft: Anesth. Analg., v. 93, p. 536-542.

All references cited herein are incorporated herein by reference in their entireties.

1. An in vitro composition comprising freeze-dried platelets and whole blood, blood plasma, or a component of whole blood or blood plasma.

2. The composition of claim 1, comprising freeze-dried platelets and fresh platelets.

3. The composition of claim 1, wherein the freeze-dried platelets are reconstituted.

4. The composition of claim 1, wherein the freeze-dried platelets are platelets from a public blood source.

5. The composition of claim 1, wherein the freeze-dried platelets are from a patient undergoing, or scheduled to undergo, a therapy that might affect blood clotting activity of that patient.

6. A method of determining the blood clotting capability of a sample comprising blood or a blood component, said method comprising
combining freeze-dried platelets with fresh blood or a blood component to create a mixture; and
assaying the mixture for one or more biological or biochemical functions indicative of one or more blood clotting functions.

7. The method of claim 6, wherein the method is a method of diagnosing a disease or disorder of the blood clotting system.

8. The method of claim 6, wherein the freeze-dried platelets are obtained from a patient having, or suspected of having, a defect in his blood clotting system.

9. The method of claim 6, wherein the fresh blood or blood component is obtained from a patient having, or suspected of having, a defect in his blood clotting system.

10. The method of claim 6, wherein the method is a method of monitoring the progression of a disease or disorder of the blood clotting system.

11. The method of claim 10, wherein the method comprises combining freeze-dried platelets obtained from a patient at a time point zero with platelets removed from the patient at a later time point to make a mixture, and determining the blood clotting ability of the mixture.

12. The method of claim 11, further comprising removing a second amount of platelets at a second later time point, combining them with the freeze-dried platelets, and determining the blood clotting ability of the mixture.

13. The method of claim 6, which is a method of monitoring the effects of a treatment regimen for a patient on the blood clotting system of that patient.

14. A kit comprising freeze-dried platelets and at least one substance that affects the blood clotting system.

15. The kit of claim 14, wherein the substance is a drug.

16. The kit of claim 15, wherein the drug has anti-platelet activity.

17. The kit of claim 15, wherein the drug reduces the ability of a patient's blood to clot.

18. The kit of claim 14, wherein the freeze-dried platelets are platelets from a public blood source.

19. The kit of claim 14, comprising multiple containers containing freeze-dried platelets.

20. The kit of claim 14, comprising multiple containers containing two or more different substances.
